(19) United States
(12) Patent Application Publication
(10) Pub. No.: US 2010/0273748 A1
(43) Pub. Date: Oct. 28, 2010

(54) ANTIMICROBIAL THERAPY

(75) Inventors: Richard L. Gallo, San Diego, CA (US); Jurgen Schauber, Munich (DE); Robert Modlin, Sherman Oaks, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/440,455

(22) PCT Filed: Sep. 7, 2007
(86) PCT No.: PCT/US07/19485

§ 371 Date: Mar. 6, 2009

Related U.S. Application Data
(60) Provisional application No. 60/843,464, filed on Sep. 8, 2006.

(51) Int. Cl. A61K 31/593; C12N 5/00; C07C 401/00; A61P 31/00; A61P 17/02

(52) U.S. Cl. 514/167; 435/375; 552/653

(57) ABSTRACT

Methods and compositions for modulating gene expression and the innate immune response by 1,25(OH)₂ vitamin D3 (1,25D3) are provided. Also provided are methods and compositions for modulating histone acetylation using 1,25(OH)₂ vitamin D3 (1,25D3).

### CROSS REFERENCE TO RELATED APPLICATIONS

This application claims priority under 35 U.S.C. §119 from Provisional Application Ser. No. 60/843,464 filed Sep. 8, 2006, the disclosure of which is incorporated herein by reference.

### STATEMENT AS TO FEDERALLY SPONSORED RESEARCH

The U.S. Government has certain rights in this disclosure pursuant to Grant No. AI052453 awarded by the National Institutes of Health.

### TECHNICAL FIELD

The disclosure relates to methods and compositions for modulating the expression or activity of factors associated with the innate immune system.

### BACKGROUND

Small, cationic antimicrobial peptides (AMPs) are naturally occurring antibiotics of the innate immune system. AMPs are widely distributed in animals and plants and are among the most ancient host defense factors. Their spectrum of activity includes Gram-positive and Gram-negative bacteria as well as fungi and certain infective agents. As resistance of pathogenic microbes to conventional antibiotics increases, researchers are exploring these endogenous antibiotics as a potential source or new therapies against variety of infectious diseases.

Infective agent infections occur following entrance of virions into host cells by a variety of mechanisms including endocytosis of non-enveloped infective agents and fusion with the cell membrane by enveloped infective agents. One primary barrier to the infection is epithelial keratinocyte of the skin. Alterations in skin barrier function are seen in atopic dermatitis (AD). This finding may contribute to infection with bacteria and selected infective agents, including Herpesviridae (herpes simplex infective agent (HSV), varicella-zoster infective agent) and vaccinia infective agent. However, it is unlikely that a defect in the physical barrier alone accounts for the remarkably increased susceptibility of AD patients to recurrent skin infections.

### SUMMARY

Treatment of skin disorders, infectious or non-infectious, with vitamin D3 can result in faster recovery from many dermatological diseases including, but not limited to, wounds, bacterial and viral infections, diabetic ulcers, chronic skin ulcers, or disorders such as acne or acne rosacea. In one aspect, compositions provided herein may include a cathelicidin (or active fragment thereof) and vitamin D3 for an antimicrobial therapy or to enhance the immune response against tumors or to accelerate wound healing.

Also provided are methods of treating atopic dermatitis. Such methods include contacting a subject having or suspected of having atopic dermatitis with an inhibiting effective amount of a cathelicidin functional fragment and/or activated vitamin D3 (1,25D3).

Also provided are methods and compositions for the treatment of skin disorders, infectious or non-infectious, with 1,25D3 such that the 1,25D3 promotes or enhances the subject's innate immune response and therefore a faster recovery from many dermatological diseases including, but not limited to, wounds, bacterial and viral infections, diabetic ulcers, chronic skin ulcers, or disorders such as acne or acne rosacea. The compositions provided herein may be utilized systemically for treatment of pneumonia, sepsis, bacterial infections, Staphylococcal aureus, TB or immunocompromised patients; or for enhancing the innate immune response in cancer treatment.

In one embodiment, a method for modulating an innate immune response in a target tissue is provided. The method includes contacting the target tissue with 1,25(OH)₂ vitamin D3. The contacting induces the expression of at least one nucleic acid sequence encoding a polypeptide that modulates the innate immune response. In one aspect, the target tissue may be contacted with 1,25(OH)₂ vitamin D3 by inducing the production of 1,25(OH)₂ vitamin D3 from a precursor molecule at the target tissue. The precursor molecule general includes vitamin D3. The production of 1,25(OH)₂ vitamin D3 may be induced by 1-alpha hydroxylase (CYP27B1) and/or 25 hydroxylase (CYP27A1).

In one aspect, the expressed nucleic acid sequence is operably associated with a regulatory sequence that includes at least one vitamin D responsive element (VDRE). The nucleic acid sequence may encode a microbial pattern recognition receptor (PRR) such as CD14 or toll-like receptor 2 (TLR2).

In other aspects, the nucleic acid sequence encodes a polypeptide that includes a cathelicidin functional peptide. The peptide may be 16-36 amino acids in length and contain the sequence NH₂—X₁X₂X₃X₄X₅X₆IKX₂FX₈X₉X₁₀LX₁₁P—COOH (SEQ ID NO:1).

The method of claim 1, wherein the target tissue comprises a skin disorder selected from a wound, a bacterial infection, a viral infection, a diabetic ulcer, a chronic skin ulcer, acne or acne rosacea.

In various embodiments described herein, the administration or contacting with 1,25(OH)₂ vitamin D3 induces expression of a nucleic acid sequence by forming a complex with the vitamin D receptor. The complex binds to a vitamin D responsive element (VDRE) associated with a regulatory element operably associated with a nucleic acid sequence encoding a polypeptide involved in the innate immune response. In some aspects, the complex further associates with VDR interacting proteins or steroid receptor co-activators (SRCs). This association promotes the modification of the histones associated with the nucleic acid sequence. More specifically, the acetylation state of the histones associated with a regulatory element operably associated with the nucleic acid sequence, is modified.

In another embodiment, a method for modulating the innate immune response of a subject is provided. The method includes administering to the subject in need of such treatment a composition, or a pharmaceutically acceptable salt thereof, containing a therapeutically effective amount of a compound. The compound includes 1,25(OH)₂ vitamin D3, a 1,25(OH)₂ vitamin D3 precursor, or an agent that increases the production of 1,25(OH)₂ vitamin D3 at the site of the target tissue. The expression of at least one nucleic acid sequence encoding a polypeptide that modulates the innate immune response is subsequently induced in the subject. In some aspects the composition further includes an antimicrobial agent such as an antibiotic. It is understood that modulating the innate immune response of a subject can effectively treat a systemic infection or localized infection.

In yet another embodiment, methods for promoting wound healing are provided. Such methods include contacting the site of a wound with 1,25(OH)₂ vitamin D3 or inducing the expression of 1,25(OH)₂ vitamin D3 at the site of the wound. In general, the contacting promotes wound healing by modulating an innate immune response in the tissue associated with the wound site.

In another embodiment, methods of increasing the expression of a microbial pattern recognition receptor (PRR) in a cell are provided. The methods include contacting the cell with a composition that includes vitamin D, 1,25(OH)₂ vitamin D3, or a compound that increases the production of 1-alpha hydroxylase (CYP27B1) in the cell. In some aspects, the microbial pattern recognition receptor (PRR) may be CD14 or toll-like receptor 2 (TLR2).

In another embodiment, a pharmaceutical composition, or pharmaceutically acceptable salt thereof, including 1,25(OH)₂ vitamin D3 or a precursor to vitamin D3, is provided. In some aspects, the pharmaceutical composition further includes an antimicrobial agent. A pharmaceutical composition provided herein may be formulated for topical administration.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a pile up of protein sequence from canine cathelicidin (SEQ ID NO:9), PR-39 (SEQ ID NO:10); LL-37 (SEQ ID NO:6), murine CRAMP (SEQ ID NO:8) and goat cathelicidin (SEQ ID NO:11).

FIG. 2 shows that carbonate containing compounds increase the antimicrobial activity of LL-37. Carbonate was identified as the antimicrobial enhancing factor in mammalian tissue culture media via serial deletion of Minimal Essential Media (MEM) components. Staphylococcus aureus was cultured in 20% Tryptic Soy Broth (TSB), 10% Fetal Bovine Serum (FBS) and 70% of various media or media components with, or without, 32 μM LL-37 to assess which components could increase antimicrobial activity. MEM buffers are 2200 mg/L NaHCO₃, 140 mg/L NaH₂PO₄ and 6800 mg/L NaCl. MEM salts are 200 mg/L CaCl₂, 400 mg/L KCl, 98 mg/L MgSO₄. All solutions were adjusted to pH 7.4. Bacterial growth was measured as the change in turbidity at OD₆₀₀ and percent inhibition was determined by comparing bacterial growth in the presence of peptide to that in the absence of peptide. 32 μM LL-37 inhibited Staphalococcus aureus growth poorly in TSB media alone, or with NaCl and FBS. In the presence of MEM buffers, inhibition was greatly increased and NaHCO₃ was determined to be the buffering component responsible for this increase. This was confirmed with other carbonate containing compounds, 50 mM KHCO₃ and Na₂CO₃, which also increased antimicrobial activity.

FIG. 3 shows that bicarbonate itself, not pH, is responsible for antimicrobial enhancement. Inhibition of S. aureus was measured in 20% TSB, 10% FBS 1 mM NaH₂PO₄, 150 mM NaCl, with either 50 mM NaHCO₃ or 50 mM glycine buffer at various pH. Only bicarbonate containing media was able to confer increased antimicrobial activity to LL-37.

FIG. 4 shows that bicarbonate enhances activity of cathelicidin antimicrobial peptides. Inhibition of S. aureus growth in the presence of various concentrations of human (LL-37), murine (CRAMP) and porcine (PR-39). Cathelicidin antimicrobial peptides were measured in 20% TSB, 1 mM NaH₂PO₄, with, or without, 50 mM NaHCO₃ at pH 7.4. The presence of NaHCO₃ greatly reduced the minimal inhibitory concentration (MIC) of all three peptides.

FIG. 5 demonstrates that 25 mM NaHCO₃ enhances LL-37 activity without effecting bacterial growth. The growth of S. aureus in the presence, or absence, of 32 μM LL-37 was measured in 20% TSB, 10% FBS, 1 mM NaH₂PO₄, 150 mM NaCl, and various concentrations of NaHCO₃, all at pH 7.4. 25 mM NaHCO₃ yielded complete inhibition of bacterial growth in the presence of LL-37, but not without peptide. High concentrations of NaHCO₃ retarded the growth of S. aureus.

FIG. 6 shows that NaHCO₃ enhances antimicrobial activity against gram negative and positive bacteria. S. aureus and E. coli were resistant to 16 uM CRAMP without 50 mM NaHCO₃, but became susceptible in its presence in 20% TSB, 1 mM NaH₂PO₄ at pH 7.4. Group B Strep (GBS) and Salmonella were susceptible even without NaHCO₃.

FIG. 7 demonstrates that NaHCO₃ enables direct membrane disruption of S. aureus. Release of cytoplasmic-expressed beta-galactosidase was measured from bacteria with and without the addition of NaHCO₃. Panel A: Polymyxin B induces greater leakage of cytoplasmic protein with carbonate. Panel B: LL-37 induces greater leakage of cytoplasmic protein with carbonate.

FIG. 8 shows that NaHCO₃ enhances the activity of various antimicrobial peptides (see also FIG. 4). The ability of 50 mM NaHCO₃ (EF) to enhance the activity of diverse antimicrobial peptides against S. aureus was tested in 20% TSB, 10% FBS, 1 mM NaH₂PO₄, 150 mM NaCl, pH 7.4. The activity of all 3 cathelicidins, Cryptdin-4 and Human Beta Defensin-2 (HBD-2) were enhanced by the addition of bicarbonate. Several other antimicrobial peptides were not enhanced.

FIG. 9 is an electron microscopy micrograph showing bacterial cell wall lysis by LL-37 and bicarbonate. S. aureus was grown for four hours in 20% TSB, 1 mM NaH₂PO₄ with or without 25 mM NaHCO₃ or 32 μM LL-37. Bacteria were fixed in Karnovsky's Solution 24 hours and processed for EM on a Zeiss EM 10B Transmission Electron Microscope. The presence of 25 mm NaHCO₃ alone had no visible effect on the bacterial cell wall. 32 μM LL-37 caused a small amount of damage to bacterial cell walls in the absence of NaHCO₃, but totally ablated them in its presence, causing cell lysis. Mag 42,000×, insets are 110,000×.

FIG. 10A shows soluble antimicrobial activity on human skin. Human sweat was concentrated 50× and separated by HPLC on C18. Panel a: Absorbance profile at 214 nm for eluted material from 35% to 60% acetonitrile, inset: complete absorbance profile of eluted materials. Panel b: Ability of material eluted in FIG. 1a to inhibit growth of S. aureus mprF is shown as diameter of zone of S. aureus mprF inhibition. Several antimicrobial fractions were detected. Mass spectrometry identified previously described antimicrobials; fractions labeled (1) and (3) are dermcidin and DCD-1L respectively (MW 4701, MW 4818, respectively) confirmed by N-terminal sequencing ((1): SSLLEKGLDGA, (3): SSLLE, (2): LL-37 identified by mass spectrometry (MW 4493) and immunoblot. Data representative of single experiment repeated 5 times with separate sweat preparations.

FIG. 11 shows serine protease in sweat enhances cathelicidin antimicrobial activity. Antibacterial activity evaluated by radial diffusion assay against S. aureus mprF after incubation of LL-37 (32 uM) in sweat. Panel a: Increase in the inhibition zone is seen when incubated in sweat but not 10 mM PBS or distilled water (DW). b) Diameters of inhibition zone after 6 hr incubation at 37° C. in (SWT buffer; sweat buffer salts alone), (PBS), (D.W.; distilled water), (SWT; sterile filtered human sweat at 37° C.), (SWT at 4° C.), (SWT with PI, with protease inhibitor cocktail). c) Action of specific protease inhibitors on gain of antimicrobial activity. Data show increase in inhibition zone at 37° C. after 6 hr compared to Ohr. Serine protease inhibitors AEBSF and Aprotinin were most effective. N.E.I.=neutrophil elastase inhibitor, L.E.I.=leukocyte elastase inhibitor. Data are triplicate determinations±SEM from single experiment representative of 3.

FIG. 12 shows purification of novel cathelicidin peptides generated from LL-37. Panel a: Human sweat was separated by HPLC on C18. Absorbance profile at 214 nm is shown for eluted material from 30% to 60% acetonitrile. Crude is sweat prior to addition of LL-37, overlay plots show separate runs of sweat following addition of 32 nmoles of LL-37 and incubation for 0 to 24 hr at 37° C. Panel b: Antibacterial activity with radial diffusion assay against S. aureus mprF of fractions eluted from 24 hr sample in Panel a. Peaks with antimicrobial activity are labeled 1 and 2 and 3.

FIG. 13 shows the identification of novel cathelicidin peptides generated from LL-37. Following purification shown in FIG. 3, major bioactive fractions were identified. Panel a: peak (1) in FIG. 12a identified as KR-20 by mass spec. (MW 2468), and N-terminal sequence KRIVQRIKDVF (SEQ ID NO:2), Panel b: peak (2) detected 2 peptides; RK-31 and KS-30 (MW 3647, 3803, and RKSKEKIG (SEQ ID NO:3), KSKEKIGK (SEQ ID NO:4), respectively). Panel c: Western blot analysis with anti-LL-37 was done on all fractions from FIG. 3, shown are fractions eluting at acetonitrile concentrations 46-55. Peaks labeled (2) and (3) in FIG. 3 were immunoreactive. Panel d: peak (3) identified as LL-37 (MW 4493). Data from single experiment representative of 3.

FIG. 14 shows the processing of LL-37 enhances antimicrobial activity. The antimicrobial activity of cathelicidin peptides were evaluated by radial diffusion assay against (a) S. aureus, (b) E. coli, (c) solution assay against C. albicans and (d) Group A Streptococci. The antimicrobial activity of each peptide was evaluated in 10% TSB/10 mM phosphate buffer with several NaCl concentrations against E. coli (e) and S. aureus (f). Data shown are representative of triplicate determinations.

FIG. 15 shows that processing of LL-37 decreases ability to stimulate keratinocyte IL-8. Cathelicidin peptides were added to culture of normal human keratinocytes at a final concentration of 3 or 10 mM then IL-8 release determined after 8 hr. All samples were endotoxin free by limulus assay. Data are mean±SEM of triplicate determinations.

FIG. 16 is a graph showing that LL-37 induces neutrophil and mast cell chemotaxis.

FIG. 17 is a graph showing that CRAMP has similar effect in mouse dendritic cells.

FIG. 18 Panels A-C show that CRAMP inhibits antigen presentation in vitro.

FIG. 19 Panels A-C shows the identification of cathelicidin function fragments on the normal skin surface by HPLC.

FIG. 20 shows a schematic of cathelicidin processing on the normal skin surface.

FIG. 21 provides data indicating that injury triggers increased TLR2, CD14 and CYP24A1 in skin. Human wounds 24 hours after full thickness sterile skin incision were evaluated for the expression of innate immune recognition and response molecules (n=5). Transcript abundance was measured by quantitative real-time PCR (qPCR), normalized to GAPDH expression, and compared to non-injured skin (n=4). Panel A: Wounded skin shows an expected increase in cathelicidin expression, a vitamin D responsive antimicrobial gene. Additional vitamin D3 responsive genes. Panel B shows the TLR co-receptor CD14 and Panel C shows the vitamin D catabolic enzyme CYP24A1, also increase after injury. Panel D: Expression of TLR2, but not TLR1, TLR4 and TLR6, is increased after injury. (E) A corresponding increase in TLR2 protein staining on keratinocytes at the wound edges is seen (displayed at 400× magnification). The incision site is marked by white arrows. (*: p<0.05; Mann-Whitney test).

FIG. 22 provides data indicating that 1,25(OH)₂ vitamin D3 induces TLR2 in keratinocytes. The effect of 1,25(OH)₂ vitamin D3 (1,25D3) on the expression of TLRs in cultured keratinocytes. Panel A: 1,25D3 (100 nM) increases expression of TLR2 and CD14 mRNA in cultured keratinocyte monolayers after 24 hours. Panel B; 1,25D3 (100 nM) increases expression of TLR2 and CD14 protein in monolayer keratinocyte extracts evaluated by Western Blot. Panel C: Keratinocytes grown in differentiated epidermal constructs stimulated with 1,25D3 (100 nM) also show an increase in CD14 and TLR2 transcript abundance. All data shown are means (±SD) of the results from a representative experiment performed in triplicate. (*: p<0.05; **: p<0.01; Student's t-test). Panel D; Skin samples from healthy volunteers (n=7) were treated with 1,25D3 (1.0 mM; once applied). Controls are contralateral skin treated with the vehicle. After 4 days punch biopsies from both sites were obtained and skin sections stained for TLR2 expression. Staining intensity increased in patients treated with topical 1,25D3 as determined by a blinded investigator graded according to the intensity of immunoreactivity (0=no expression; 3=strong expression). Sections from one representative study participant are displayed. (*: p<0.05; Mann-Whitney test)

FIG. 23 provides data indicating that CYP27B1 is increased following injury, TGF or activation of TLR2. Panel A: The expression of CYP27B1, which converts inactive 25D3 to active 1,25D3, was evaluated in skin wounds as in FIG. 21. Wounded skin (n=5) increased CYP27B1 mRNA compared to controls (n=4). (*: p<0.05; Mann-Whitney test). Panel B: Keratinocytes were cultured in the presence of TNFα (20 ng/ml) or TGF (1 μg/ml) for 24 hours then RNA isolated and CYP27B1 transcript abundance analyzed by qPCR. Panel C; Keratinocytes were cultured with different TLR ligands for 24 hours cells then CYP27B1 expression analyzed as above. Both TGF and the TLR2 ligand Malp-2 induced CYP27B1 expression. Data shown are means (±SD) of a single experiment performed in triplicate and is representative of three independent experiments. (*: p<0.05; **: p<0.01; Student's t-test)

FIG. 24 provides data indicating that TGF expression leads to an increase of 1,25D3 responsive genes found in wounds by activation of CYP27B1. Keratinocytes were pretreated with the VDR antagonist VAZ (VDR-antagonist ZK159222; 10⁻⁷M) or the CYP27B1 antagonist itraconazole (ITRA; 10⁻⁷M) and then stimulated with TGF (1 μg/ml) in the presence or absence of 25D3 (10 nM) for 24 hours. Panel A: Expression of cathelicidin mRNA. Panel B shows CD14 mRNA, and Panel C shows TLR2 mRNA determined as described in FIG. 22. Induction of these innate immune effectors and response genes by TGFβ as found in wounds is dependent on the availability of 25D3 and the activity of CYP27B1 or a functional VDR. Data shown are means (±SD) of the results from a single experiment performed in triplicate and are representative of three independent experiments. (*: p<0.05; Student's t-test).

FIG. 25 provides data indicating that activation of CYP27B1 is responsible for increased vitamin D3 signaling in wounds. Wounds from CYP27B1 −/− and age matched wild type (WT) littermates 24 hours after full thickness sterile skin incision were evaluated for the expression of innate immune recognition and response molecules (n=5 per group). Transcript abundance in wounded skin was measured by quantitative real-time PCR (qPCR) and normalized to non-injured skin from the same animal. Panel A: CD14—a 1,25D3 regulated gene in mice—increases in wild type animals after injury but not in mice lacking CYP27B1. Panel B: Murine cathelicidin, which is not regulated by 1,25D3, shows increased expression in wounds in both animal strains. (*: p<0.05; **: p<0.01; Mann-Whitney test)

FIG. 26 provides data indicating that 1,25D3 increases TLR2 function in keratinocytes. Panel A: To test the functional relevance of the increased expression of TLR2 and CD14 in keratinocytes exposed to 1,25D3, cells were incubated with low dose 1,25D3 (0.1 nM, 24 hours) then stimulated with TLR ligands for an additional 24 hours. Cathelicidin mRNA abundance was measured as in FIG. 22. Only TLR2/6 ligand Malp-2 increased expression above that induced by low dose 1,25D3 alone. Panel B; Dose dependent response of cathelicidin expression following TLR2,6 agonists Malp-2, FSL-1 or zymosan at the indicated concentrations in the presence of 1,25D3 (100 nM). Panel C: Cathelicidin mRNA expression in keratinocytes incubated with low dose 1,25D3 (0.1 nM, 24 hours) or high calcium (1.7 mM) for 24 hours, and then stimulated with Malp-2 for another 24 hours. Panel D: Organotypic epidermal constructs were stimulated with Malp-2 (0.1 μg/ml) alone, or in the presence of 1,25D3 (100 nM), and cathelicidin peptide expression determined by immunostaining. Constructs were stained with a polyclonal anti-LL-37 antibody (green) and nuclei detected with DAPI (blue) (400× magnification). Panel E: Cathelicidin mRNA expression is inhibited by a TLR2 neutralizing antibody (aTLR2) applied to keratinocytes stimulated with Malp-2 or FSL-1 in the presence of 1,25D3 (100 nM). Panel F: A functional VDRE is required for transcriptional activation by Malp-2 and 1,25 D3. HaCat keratinocytes containing cathelicidin promoter reporter constructs (pGL3 1500), and treated with Malp-2 (0.1 μg/ml) in the presence or absence of 1,25D3 (100 nM). Promoter constructs with a deleted VDRE at position -619 bp to −633 bp (pGL3 1500-VDRE) lost transcriptional activity in all experiments. Data shown are means (±SD) of a single experiment performed in triplicate and are representative of three independent experiments. (*: p<0.05; **: p<0.01; ***: p<0.001; Student's t-test).

FIG. 27 provides a schematic model for 1,25D3-regulated innate immune functions in keratinocytes and monocytes. Two distinct 1,25D3-dependent pathways in keratinocytes and monocytes are shown. In skin injury, keratinocytes are activated by TGF-β1 or TLR2/6 ligands, which then leads to induction of CYP27B1. As a consequence 25D3 is converted to 1,25D3, which, upon activation of the VDR, induces cathelicidin, TLR2, and CD14. The 1,25D3-induced TLR2 enables the response of keratinocytes to TLR2 activation, resulting in further increased cathelicidin expression. In contrast, circulating monocytes are activated by TLR2/1 agonists. As a consequence, the genes encoding the VDR and CYP27B1 are induced. CYP27B1 converts 25D3 to 1,25D3 and subsequently increases cathelicidin.

FIG. 28 provides data indicating that SRC3 expression can be localized to skin layers that express 1,25D3-dependent innate defense genes. Differential localization of DRIP205 (see panel a) and SRC3 (see panel b) in the epidermis. Human adult skin sections were incubated with antibodies against DRIP205 and SRC3, and the signals were visualized with biotinylated secondary antibody and NBT/BCIP staining. The nuclei were counterstained with nuclear fast red (DRIP205 and SRC3). DRIP localizes to nuclei in the basal and suprabasal layer, whereas SRC3 is expressed in differentiated keratinocytes in the outmost epidermis. Panel c and panel d depict corresponding sections stained with the respective pre-immune control antibodies. Bar=35 μM.

FIG. 29 provides data indicating that HDAC inhibition increases 1,25D3-mediated induction of cathelicidin antimicrobial peptide in human keratinocytes. Panel a: NHEKs were stimulated with 1,25D3 (10⁻⁹-10⁻⁷ M) in the presence of the HDAC inhibitor butyrate (BUT; 2 mM) for 24 hours. mRNA abundance was determined by real-time qPCR for cathelicidin and glyceraldehyde-3-phosphate dehydrogenase and normalized to vehicle-treated controls. BUT alone had no effect, whereas HDAC inhibition strongly increased 1,25D3-induced cathelicidin. Data shown are means (7SD) of the results from a single stimulation experiment performed in triplicates and are representative of at least three independent experiments (**P<0.01; ***P<0.001; two-way analysis of variance). Panel b: The HDAC inhibitor TSA (200 ng ml⁻¹) induces cathelicidin in the presence of 1,25D3 (10⁻⁸M). Expression of cathelicidin is dependent on a functional VDR, as pretreatment with the VDR antagonist ZK159222 (10⁻⁷ M) inhibits induction of cathelicidin with BUT (2 mM) in the presence of 1,25D3 (10⁻⁸ M) after 24 hours. Expression of cathelicidin was determined by qPCR as described in Panel a. (*P<0.05; Student's t-test). Panel c: Increased transcript correlates with enhanced cathelicidin protein expression after HDAC inhibitor and 1,25D3 stimulation. NHEKs grown on chamber slides were stimulated with the vehicle, 1,25D3 (10⁻⁸M), BUT (2 mM), or the combination for 24 hours. Cells were stained with a polyclonal anti-LL-37 antibody (followed by an FITC-coupled secondary antibody) and nuclei detected with 4,6-diamidino-2-phenylindole (Bar=30 μM). Panel d: Enhanced antimicrobial activity of keratinocytes after BUT and 1,25D3 stimulation. NHEKs were stimulated with 1,25D3 (10⁻⁸M) in combination with the HDAC inhibitor BUT (2 mM) for 24 hours, cells harvested and cell lysates co-incubated with S. aureus DmprF, and bacterial growth monitored over time to determine antimicrobial activity. Media not containing cell lysates or containing cell lysates from unstimulated cells were used as controls. OD₆₀₀ readings after 6 hours incubation are displayed (*P<0.05; Student's t-test).

FIG. 30 provides data indicating that HDAC inhibition in keratinocytes increases CD14, CYP24A1, and cathelicidin, but not IL-8. Panel a: NHEKs were stimulated with 1,25D3 (10⁻⁸M), in the presence of butyrate (BUT; 2 mM) for 24 hours. Transcript abundance of 1,25D3-regulated CD14 and CYP24A1 was determined by qPCR (**P<0.01; Student's t-test). Panel b: (Upper panel) Western blot of CD14 protein expression in NHEK stimulated with control (lane 1), 1,25D3 (10⁻⁸M; lane 2), BUT (2 mM; lane 3), or the combination of 1,25D3 and BUT (lane 4) for 24 hours. A band at approximately 50 kDa was detected corresponding to CD14 protein. Membranes were re-probed with an anti-alpha tubulin antibody and protein abundance quantified by densitometry (right panel). Panel c: NHEKs stimulated with 1,25D3 (10⁻⁸M), the TLR2/6 ligand Malp-2 (0.1 μg ml⁻¹), or the combination, and cathelicidin or IL-8 transcript abundance determined by qPCR. Panel d: HDAC inhibition by BUT did not enhance IL-8 transcript induced by Malp-2.

FIG. 31 provides data indicating that HDACi and TLR activation induce histone acetylation. Panel a; Immunofluorescence staining of acetylated histone H4 protein in NHEK treated with HDACi in the presence or absence of 1,25D3. Cells treated with the combination of 1,25D3 and HDACi show increased histone acetylation. Panel b: Western blot of acetylated histone 4 from keratinocytes treated with the HDACi butyrate or TLR agonists. Panel c: Butyrate or stimulation with TLR2 ligands (Malp-2 or PAM3CSK) increased histone H4 protein acetylation. Bar=30 μM.

FIG. 32 provides data indicating that increased cathelicidin by 1,25D3 is dependent on the VDR and VDR co-activator SRC3 in keratinocytes. Panel a: Silencing of the VDR blocks cathelicidin induction in keratinocytes. NHEKs were transfected with siRNA oligonucleotides for VDR, DRIP205, SRC3, or control siRNA and stimulated with increasing concentrations of 1,25D3. Expression was normalized to the housekeeping gene L19, which is unaffected by siRNA transfection. Panel b: Efficiency of siRNA silencing was evaluated by western analysis. Panel c: siRNA suppression of the VDR co-activator SRC3, but not DRIP205, blocked induction of cathelicidin by 1,25D3. Panel d: NHEK transfected with siRNA oligonucleotides for SRC3 or DRIP205 and stimulated with 1,25D3 (10⁻⁸M), butyrate (2 mM), or the combination. Again, silencing of SRC3 blocks induction of cathelicidin and CD14, whereas silencing of DRIP205 has no effect. HDAC inhibition by butyrate is not sufficient to reverse this effect.

### DETAILED DESCRIPTION

So long as there is an apparent need for protection against infection the evaluation of host responses that contribute to control of bacterial and viral infections in general is an important goal. The disclosure provides peptides useful in treating bacterial, viral and other microbial infections.

Provided herein are methods for modulating the innate immune response of a subject. The methods include administering to the subject in need of such treatment a composition, or a pharmaceutically acceptable salt thereof, containing a therapeutically effective amount of a compound. The compound includes 1,25(OH)₂ vitamin D3, a 1,25(OH)₂ vitamin D3 precursor, or an agent that increases the production of 1,25(OH)₂ vitamin D3 at the site of the target tissue. The expression of at least one nucleic acid sequence encoding a polypeptide that modulates the innate immune response is subsequently induced in the subject. In some aspects the composition further includes an antimicrobial agent such as an antibiotic. It is understood that modulating the innate immune response of a subject can effectively treat a systemic infection or localized infection.

Also provided are methods for promoting wound healing. Such methods include contacting the site of a wound with 1,25(OH)₂ vitamin D3 or inducing the expression of 1,25(OH)₂ vitamin D3 at the site of the wound. In general, the contacting promotes wound healing by modulating an innate immune response in the tissue associated with the wound site.

Also provided are methods of increasing the expression of a microbial pattern recognition receptor (PRR) in a cell. The methods include contacting the cell with a composition that includes vitamin D, 1,25(OH)₂ vitamin D3, or a compound that increases the production of 1-alpha hydroxylase (CYP27B1) in the cell. In some aspects, the microbial pattern recognition receptor (PRR) may be CD14 or toll-like receptor 2 (TLR2).

Also provided are pharmaceutical compositions, or pharmaceutically acceptable salts thereof, that include 1,25(OH)₂ vitamin D3 or a precursor to vitamin D3. In some aspects, the pharmaceutical composition further includes an antimicrobial agent. A pharmaceutical composition provided herein may be formulated for topical administration.

“Innate immunity” refers to the innate immune system, which, unlike the “adaptive immune system”, uses a set of germline-encoded receptors for the recognition of conserved molecular patterns present in microorganisms. The innate immune system provides the body with a first line defense against invading pathogens. In an innate immune response, an invading pathogen is recognized by a germline-encoded receptor, the activation of which initiates a signaling cascade that leads to the induction of cytokine expression. Innate immune system receptors have broad specificity, recognizing molecular structures that are highly conserved among different pathogens. These receptors are known as Toll-like receptors (TLRs), due to their homology with receptors that were first identified and named in Drosophila, and are present in cells such as macrophages, dendritic cells, and epithelial cells.

“Toll-like receptor” (TLR) refers to any of a family of receptor proteins that are homologous to the Drosophila melanogaster Toll protein. TLRs also refer to type I transmembrane signaling receptor proteins that are characterized by an extra-cellular leucine-rich repeat domain and an intracellular domain homologous to that of the interleukin 1 receptor. The TLR family includes, but is not limited to, mouse TLR2 and TLR4 and their homologues, particularly in other species including humans. There are at least ten different TLRs in mammals, and ligands and corresponding signaling cascades have been identified for some of these receptors. For example, TLR2 is activated by the lipoprotein of bacteria (e.g., E. coli), TLR3 is activated by double-stranded RNA, TLR4 is activated by lipopolysaccharide (i.e., LPS or endotoxin) of Gram-negative bacteria (e.g., Salmonella and E. coli O157:H7), TLR5 is activated by flagellin of motile bacteria (e.g., Listeria), and TLR9 is activated by unmethylated CpG sequences of pathogen DNA. The stimulation of each of these receptors leads to activation of the transcription factor NF-kappa B, and other signaling molecules that are involved in regulating the expression of cytokine genes, including those encoding tumor necrosis factor-alpha (TNF-alpha), interleukin-1 (IL-1), and certain chemokines.

“Molecular pattern” refers to a chemical structure or motif that is typically a component of microorganisms, or certain other organisms, but which is not typically produced by normal human cells or by other normal animal cells. Molecular patterns are found in, or composed of, the following types of molecules: lipopolysaccharides, peptidoglycans, lipoteichoic acids, phosphatidyl cholines, lipoproteins, bacterial DNAs, viral single and double-stranded RNAs, certain viral glycoproteins, unmethylated CpG-DNAs, mannans, and a variety of other bacterial, fungal and yeast cell wall components and the like.

“Pattern Recognition Receptor” or “PRR” refers to a member of a family of receptors of the innate immune system that, upon binding a PAMP, an immunostimulatory portion or derivative thereof, can initiate an innate immune response. Members of this receptor family are structurally different and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly. Cellular PRRs may be expressed on effector cells of the innate immune system, including cells that function as professional APCs in adaptive immunity, and also on cells such as surface epithelia that are the first to encounter pathogens during infection. PRRs may also induce the expression of a set of endogenous signals, such as inflammatory cytokines and chemokines. Examples of PRRs include, but are not limited to, the following: C-type lectins (e.g., humoral, such as collectins (MBL), and cellular, such as macrophage C-type lectins, macrophage mannose receptors, DEC205); proteins containing leucine-rich repeats (e.g., Toll receptor and TLRs, CD14, RP105); scavenger receptors (e.g., macrophage scavenger receptors, MARCO, WCI); and pentraxins (e.g., C-reactive proteins, serum, amyloid P, LBP, BPIP, CD11b, C and CD18.

“Pathogen-Associated Molecular Patten” or “PAMP” refers to a molecular pattern found in a microorganism but not in humans, which, when it binds a PRR, can trigger an innate immune response. Thus, as used herein, the term “PAMP” includes any such microbial molecular pattern and is not limited to those associated with pathogenic microorganisms or microbes. As used herein, the term “PAMP” includes a PAMP, derivative or portion of a PAMP that is immunostimulatory, and any immunostimulatory molecule derived from any PAMP. These structures, or derivatives thereof, are potential initiators of innate immune responses, and therefore, ligands for PRRs, including Toll receptors and TLRs. “PAMPs” are immunostimulatory structures that are found in, or composed of molecules including, but not limited to, lipopolysaccharides; phosphatidyl choline; glycans, including peptidoglycans; teichoic acids, including lipoteichoic acids; proteins, including lipoproteins and lipopeptides; outer membrane proteins (OMPs), outer surface proteins (OSPs) and other protein components of the bacterial cell walls and Flagellins; chaperones including periplasmic chaperones such as FimC; bacterial DNAs; single and double-stranded viral RNAs; unmethylated CpG-DNAs; mannans; mycobacterial membranes; porins; and a variety of other bacterial and fungal cell wall components, including those found in yeast.

Compositions provided herein can be used concurrently with other antibacterial agents including sulfa drugs such as sulfamethizole, sulfisoxazole, sulfamonomethoxine, sulfamethizole, salazosulfapyridine, silver sulfadiazine and the like; quinoline antibacterial agents such as nalidixic acid, pipemidic acid trihydrate, enoxacin, norfloxacin, ofloxacin, tosufloxacin tosilate, ciprofloxacin hydrochloride, lomefloxacin hydrochloride, sparfloxacin, fleroxacin and the like; antiphthisics such as isoniazid, ethambutol (ethambutol hydrochloride), p-aminosalicylic acid (calcium p-aminosalicylate), pyrazinamide, ethionamide, protionamide, rifampicin, streptomycin sulfate, kanamycin sulfate, cycloserine and the like; antiacid fast bacterium drugs such as diaphenylsulfone, rifampicin and the like; antiviral drugs such as idoxuridine, acyclovir, vidarabine, ganciclovir and the like; anti-HIV agents such as zidovudine, didanosine, zalcitabine, indinavir sulfate ethanolate, ritonavir and the like; antispirocheteles; antibiotics such as tetracycline hydrochloride, ampicillin, piperacillin, gentamicin, dibekacin, kanendomycin, lividomycin, tobramycin, amikacin, fradiomycin, sisomycin, tetracycline, oxytetracycline, rolitetracycline, doxycycline, ampicillin, piperacillin, ticarcillin, cephalothin, cephapirin, cephaloridine, cefaclor, cephalexin, cefroxadine, cefadroxil, cefamandole, cefotoam, cefuroxime, cefotiam, cefotiam hexetil, cefuroxime axetil, cefdinir, cefditoren pivoxil, ceftazidime, cefpiramide, cefsulodin, cefinenoxime, cefpodoxime proxetil, cefpirome, cefozopran, cefepime, cefsulodin, cefinenoxime, cefinetazole, cefminox, cefoxitin, cefbuperazone, latamoxef, flomoxef, cefazolin, cefotaxime, cefoperazone, ceftizoxime, moxalactam, thienamycin, sulfazecin, aztreonam or a salt thereof, griseofulvin, lankacidin-group and the like.

In humans, there are several classes of known antimicrobial peptides (AMPs) including α-defensins, β-defensins, and cathelicidins. Cathelicidins are found in several mammalian species. Production of cathelicidins is induced in response to epithelial wounding or infectious challenge, or suppressed by the virulence mechanisms of certain bacterial pathogens, e.g., Shigella dysenteriae. Cathelicidin expression is also differentially effected in certain chronic inflammatory disorders. In psoriasis, cathelicidin levels are elevated and secondary infection is rare, whereas in atopic dermatitis cathelicidin expression is deficient and bacterial or viral superinfection is common. Therapeutic benefits of cathelicidin have been demonstrated experimentally, including decreased bacterial colonization of skin wounds following topical administration and improved pulmonary bacterial clearance with cathelicidin overexpression through viral gene transfer.

Vitamin D3 (or its analogs) or Vitamin D3 (or its analog) in combination with a cathelicidin can be administered systemically to treat systemic infections, in particular pneumonia, sepsis and TB. It may also be applied topically to treat infectious skin disorders. It may be used in combination therapy with antibiotics or to treat immunocompromised patients such as HIV positive individuals. In combination with immune stimulating approaches, it may therapeutically address cancer.

The invention demonstrates that vitamin D3 metabolism increases during wound repair, leading to expression of cathelicidin (antimicrobial peptide). Vitamin D3 also leads to increased expression of Toll-like receptor 2 (TLR2) and CD14, which in turn induce antimicrobial peptides. Thus, Vitamin D3 both induces cathelicidin and enables TLR2 responsiveness to further increase expression of cathelicidin. This leads to enhanced innate immunity and protection against infection. It logically follows that treatment of skin disorders, infectious or non-infectious, with VD3 would result in an enhanced innate immune response. Normal keratinocytes stimulated with Vitamin D3 show enhanced antimicrobial activity over unstimulated keratinocytes against Staphalococcus aureus. Vitamin D3 induced the expression of cathelicidin in normal human keratinocytes as well as the keratinocytic cell line HaCat. Vitamin D3 response element in the cathelicidin promoter was necessary for the antimicrobial response. The role of Vitamin D3 in wound repair was previously unknown and unsuspected.

Microbial recognition through Toll-like receptors (TLRs), and an antimicrobial response through the release of antimicrobial peptides, are essential elements of the innate immune response.

The innate immune response comprises all mechanisms that resist infection but does not require prior exposure to the pathogen. Although an evolutionarily ancient system, progress in this field continues to show that the rapid innate response is an important part of mammalian immunity and can explain human disease. For example, recent insights into the functions of 1,25(OH)₂ vitamin D3 (1,25D3) as an immune modifying agent have illuminated a large body of previously unexplained associations between alterations in vitamin D3 and infectious disease. Elevated 1,25D3 and hypercalcemia has been associated with active pulmonary tuberculosis, and lower serum concentrations of the 1,25D3 precursor 250H vitamin D3 (25D3) in African Americans correlates with increased susceptibility to infection. An explanation for these events has been provided by observations that stimulation of TLR2 will increase production of 1,25D3, and that this in turn leads to an increase in the production of antimicrobial peptides (AMPs). The invention demonstrates a role for vitamin D3 in the regulation of innate immune functions.

Findings that 1,25D3 regulates the expression and activation of AMPs in monocytes and at epithelial surfaces such as the epidermis suggest, that in addition to its effects on differentiation and formation of a physical barrier, 1,25D3 also provides a stimulus for rapid production of a chemical antimicrobial shield. In particular, 1,25D3 induces the expression of LL-37, a human AMP belonging to the cathelicidin family. These AMPs are relevant to defense against microbes as infections develop more easily in mice deficient in the cathelicidin gene or humans with a deficiency in cathelicidin expression. With the observation that cathelicidin is increased with an increase in 1,25D3, the importance of vitamin D3 to immune defense warrants renewed interest.

Vitamin D3 is produced from dietary or endogenous precursors under the influence of UVB light. Activation of vitamin D3 to 1,25D3 requires two major hydroxylation steps, the first by 25-hydroxylase (CYP27A1) and then by 1α-hydroxylase (CYP27B1). These enzymes are mainly located in the human liver and kidney, respectively. However, some 1,25D3 targeted organs such as the epidermis also posses the enzymes to produce 1,25D3. Upon binding to the vitamin D receptor (VDR) 1,25D3 activates target genes through vitamin D responsive elements in the gene promoter. Simultaneously, 1,25D3 induces the vitamin D3 catabolic enzyme CYP24A1 (24-hydroxylase) thereby initializing its own degradation. Control of 1,25D3 producing and catabolizing enzymes therefore determines the level of bioactive hormone.

Control of cathelicidin expression has followed a pattern consistent with expectations for a gene required for innate immune response, but these events are not known to involve alterations in 1,25D3. Cathelicidin, and several other AMPs greatly increase after wounding or infection, but most classical signalling molecules active in the wound repair response, or microbial ligands that trigger pattern recognition events, have little or no effect on cathelicidin expression. From observations that cathelicidin is induced by 1,25D3 in vitro, the invention demonstrates that vitamin D3 signalling is activated during skin injury. The experiments presented herein demonstrate expression of genes influenced by 1,25D3 and correlated with events observed during wound repair. The data demonstrate the induction of 1,25D3 regulated genes and that elements of innate immunity not known to change with injury are both induced by this event and under control of 1,25D3. Furthermore, the invention demonstrates that factors important to repair have an unsuspected role in modifying vitamin D3 metabolism. Thus, the invention presents data in support of a previously unknown role for vitamin D3 in wound repair and provides new insight into factors important to the control of the innate immune response.

Vitamin D3 and its hormonally active forms are included within the scope of the invention. As used herein “vitamin D3 compounds” or “1,25D3” (also referred to herein as “D3 compounds”) is intended to include compounds which are structurally similar to vitamin D3. Many of these compounds are art-recognized and comprise a large number of natural precursors, metabolites, as well as synthetic analogs of the hormonally active 1,25-dihydroxyvitamin D3 (1α25(OH)₂D3). This language is intended to include vitamin D3, or an analog thereof, at any stage of its metabolism, as well as mixtures of different metabolic forms of vitamin D3 or analogs thereof. Furthermore, the term “vitamin D3 compound” or “1,25D3” also includes synthetic analogs of vitamin D3.

Also useful in the methods and compositions of the invention are vitamin D3 receptor agonists. Examples of such agonist include biphenyl receptor agonist, such as those disclosed in U.S. Patent publication no. 20060025474.

1,25D3 hydroxylase, and specific receptors in several tissues, capable of converting non-active vitamin D3 to active 1,25D3 are found in such tissues as bone, keratinocytes, placenta, and immune cell.

Cathelicidin proteins are composed of two distinct domains: an N-terminal “cathelin-like” or “prosequence” domain and the C-terminal domain of the mature AMP. The C-terminal domains of cathelicidins were among the earliest mammalian AMPs to show potent, rapid, and broad-spectrum killing activity. The term “cathelin-like” derives from the similarity of the N-terminal sequence with that of cathelin, a 12 kDa protein isolated from porcine neutrophils that shares similarity with the cystatin superfamily of cysteine protease inhibitors.

Cathelicidins are expressed in neutrophils and myeloid bone marrow cells and most epithelial sources, and were the first AMPs discovered in mammalian skin due to their presence in wound fluid. In the neutrophil, cathelicidins are synthesized as full-length precursor and targeted to the secondary granules where they are stored. Upon stimulation, the full-length cathelicidin protein is proteolytically processed to unleash the microbiocidal activity of the C-terminal peptide from the cathelin-like domain.

The C-terminal 37 amino acids of human cathelicidin (LL-37) has been characterized. LL-37 was originally referred to as FALL39, named for the first 4 N-terminal amino acids of this domain and the total number of residues (i.e., 39). LL-37 is a peptide predicted to contain an amphipathic alpha helix and lacks cysteine, making it different from all other previously isolated human peptide antibiotics of the defensin family, each of which contain 3 disulfide bridges. Full length human cathelicidin (sometimes referred to as full length LL-37) comprises the cathelin-like precursor protein and the C-terminal LL-37 peptide, thus comprising 170 amino acids (SEQ ID NO:6).

The polypeptide comprising SEQ ID NO:6 has a number of distinct domains. For example, a signal domain comprising a sequence as set forth from about 1 to about 29-31 of SEQ ID NO:6 is present. The signal domain is typically cleaved following amino acid number 30 of SEQ ID NO:6, however, one of skill in the art will recognize that depending upon the enzyme used, the expression system used and/or the conditions under which proteolytic cleavage of the polypeptide takes place, the cleavage site may vary from 1 to 3 amino acid in either direction of amino acid number 30 of SEQ ID NO:6. Another domain comprises the N-terminal domain, referred to as the cathelin-like domain. The cathelin-like domain comprises from about amino acid 29 (e.g., 29-31) to about amino acid 128 (e.g., 128-131) of SEQ ID NO:6. Yet another domain of SEQ ID NO:6 comprises the C-terminal domain referred to as LL-37. The LL-37 domain comprises from about amino acid 128 (e.g., 128-134) to amino acid 170 of SEQ ID NO:6.

LL-37 comprises the amino acid sequence set forth in SEQ ID NO:6. The human cDNA sequence for LL-37 is set forth in SEQ ID NO:5. The coding sequence of an active fragment of LL-37 can be identified with reference to the cDNA sequence provided in SEQ ID NO:5 without difficulty. Accordingly the corresponding coding sequences of the fragments identified herein are also provided by the disclosure.

The mechanisms by which cationic human antimicrobial peptides kill bacteria and fungi are generally through binding of the peptide to the microbial cell membrane, after which the membrane's proton gradient and integrity are lost.

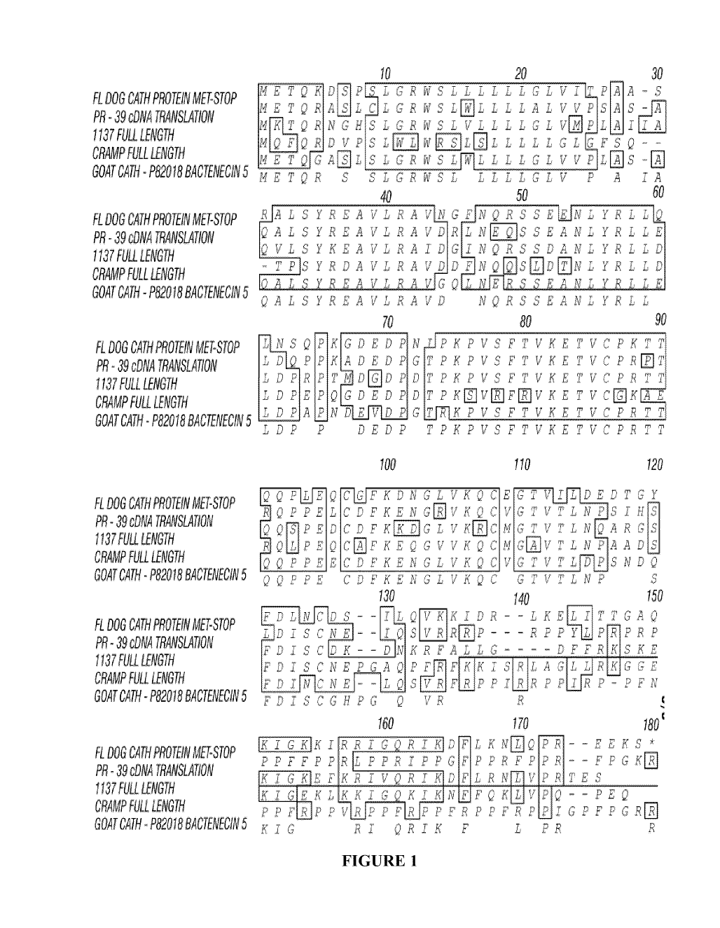

The compositions and methods of the invention utilize Vitamin D3 alone or in combination with antimicrobial peptides including cathelicidins and functional fragments thereof. Thus, a peptide useful in the methods and compositions of the invention can comprise a found in a number of peptides which inhibit bacterial growth and/or viral growth and mimic the activity of LL-37, CRAMP, and/or FALL-39. This consensus sequence is 18 amino acids in length and is found in LL-37, CRAMP and FALL-39. The consensus sequences corresponds to amino acids 151-166 of full-length LL-37 (SEQ ID NO:6), and amino acids 154-169 of CRAMP (SEQ ID NO:8). The peptides of the invention consist of 16-36 amino acids and contain NH₂—X₁X₂X₃X₄X₅X₆IKX₇FX₈X₉X₁₀LX₁₁X₁₂—COOH (SEQ ID NO:1), wherein X₄, X₂, and X₆ are individually K or R; wherein X₃ is I or K; wherein X₄ is V or G; wherein X₅ is Q or R; wherein X₇ is any amino acid; wherein X₈ is L or F; wherein Xₐ is R or Q; X₁₀ is N or K; X₁₁ is V or A; and wherein X₁₂ is P or L. A pile up of related cathelicidin proteins is provided in FIG. 1.

In one aspect, the invention provides active fragments of LL-37 and CRAMP that inhibit bacterial growth and viral infection. For example, active fragments of LL-37 include SEQ ID NO:6 from about amino acid XX₁ to XX₂, wherein XX₁ is an amino acid between and including 135 and 151 (i.e., 135, 136, 137, 138, 139, 140, 141, 142, 143, 144, 145, 146, 147, 148, 149, 150, and 151) and wherein XX₂ is an amino acid between and including 166 and 170 (i.e., 166, 167, 168, 169, and 170). Table 1 sets forth exemplary sequence of cathelicidin functional fragments.

TABLE 1

| | SEQ ID NO: 6 |
|---|---|
| Sequence | from aa to aa |
| SKEKIGKEFKRIVQRIKDFLRNLVPRTES | 1 to 170 |
| | (SEQ ID NO: 27) |
| KEKIGKEFKRIVQRIKDFLRNLVPRTES | 2 to 170 |
| | (SEQ ID NO: 22) |
| IVQRIKDFLRNLVPRTES | 1 to 170 |
| | (SEQ ID NO: 17) |

The pile up alignments of FIG. 1 are useful in identifying amino acids that may be substituted (i.e., that are not highly conserved) in peptide that can be used in combination with vitamin D3. For example, with reference to FIG. 1 it will be apparent that bolded amino acids are conserved compared to the amino acids presented in regular text. Accordingly, substitutions of amino acid at places in the peptide in regular text will likely be tolerated in contrast to non-conservative substitutions of bolded amino acids.

The term “antimicrobial” as used herein means that the methods and compositions are sufficient to destroy, or inhibit or prevent the growth or proliferation of, a microbe (e.g., a bacterium, fungus, and/or infective agent). Likewise, the term “antiviral” as used herein means that a composition or method provided herein is sufficient to destroy, or inhibit or prevent the growth or proliferation of an infective agent or an infective agent-infected cell. The term “anti-tumor” as used herein means that a composition or method of the invention prevents, inhibits the growth of, or destroys, a tumor cell(s). Similarly, the term “antifungal” means that a composition or method of the invention prevents, destroys, or inhibits the growth of a fungus.

As used herein, the term “cathelicidin functional fragment” refers to a chain of amino acids containing a cathelicidin amino acid sequence sufficient to have antimicrobial activity, e.g., that is about 16 to 36 amino acids in length and (i) contains a sequence as set forth in SEQ ID NO:1, (ii) is a fragment of the C-terminus of SEQ ID NO:6 containing SEQ ID NO:1, (iii) is an active fragment of LL-37 from about amino acid XX₁ to XX₂ of SEQ ID NO:6, wherein XX₁ is an amino acid between and including 135 and 151 (i.e., 135, 136, 137, 138, 139, 140, 141, 142, 143, 144, 145, 146, 147, 148, 149, 150, and 151) and wherein XX₂ is an amino acid between and including 166 and 170 (i.e., 166, 167, 168, 169, and 170), or (iv) a fragment set forth in Table 1. A peptide is “cationic” if it has a pKa greater than 9.0. Typically, at least four of the amino acid residues of the cationic peptide are positively charged residues, e.g., lysine and arginine. “Positively charged” refers to the side chain of an amino acid residue that has a net positive charge at pH 7.0.

The term “purified” and “substantially purified” as used herein refers to a polypeptide or peptide that is substantially free of other proteins, lipids, and polynucleotides (e.g., cellular components with which an in vivo-produced polypeptide or peptide would naturally be associated). Typically, the peptide is at least 70%, 80%, or most commonly 90% pure by weight.

The disclosure also includes analogs, derivatives, conservative variations, and cathelicidin functional fragment variants of the enumerated cathelicidin functional fragments, provided that the analog, derivative, conservative variation, or variant has a detectable antimicrobial antibacterial and/or antiviral activity. It is not necessary that the analog, derivative, variation, or variant have activity identical to the activity of the peptide from which the analog, derivative, conservative variation, or variant is derived. For example, using the alignment provided in FIG. 1, one of skill in the art can readily identify conserved amino acids and non-conserved amino acid. Using the alignment, one of skill in the art can readily identify which amino acid may be modified or substituted.

A cathelicidin functional fragment “variant” is an antimicrobial, antibacterial and/or antiviral peptide that is an altered form of a referenced cathelicidin functional fragment. For example, the term “variant” includes a cathelicidin functional fragment produced by the method disclosed herein in which at least one amino acid (e.g., from about 1 to 10 amino acids) of a reference peptide is substituted with another amino acid. The term “reference” peptide means any of the cathelicidin functional fragments of the disclosure (e.g., as defined in the above formula (SEQ ID NO:1) as well as the specific fragments described in Table 1 from which a variant, derivative, analog, or conservative variation is derived. Included within the term “derivative” is a hybrid peptide that includes at least a portion of each of two cathelicidin functional fragments (e.g., 30-80% of each of two cathelicidin functional fragments). Derivatives can be produced by adding one or a few (e.g., 1-5) amino acids to a peptide of the disclosure without completely inhibiting the activity of the peptide. In addition, C-terminal derivatives, e.g., C-terminal methyl esters, can be produced and are encompassed by the disclosure.

The disclosure also includes peptides that are conservative variations of those peptides as exemplified herein. The term “conservative variation” as used herein denotes a peptide or polypeptide in which at least one amino acid is replaced by another, biologically similar residue. Examples of conservative variations include the substitution of one hydrophobic residue, such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acid, or glutamine for asparagine, and the like. Neutral hydrophilic amino acids that can be substituted for one another include asparagine, glutamine, serine and threonine. The term “conservative variation” also encompasses a peptide having a substituted amino acid in place of an unsubstituted parent amino acid; typically, antibodies raised to the substituted peptide or polypeptide also specifically bind the unsubstituted peptide or polypeptide.

Peptides of the disclosure can be synthesized by commonly used methods such as those that include t-BOC or FMOC protection of alpha-amino groups. Both methods involve stepwise synthesis in which a single amino acid is added at each step starting from the C terminus of the peptide (See, Coligan, et al., Current Protocols in Immunology, Wiley Interscience, 1991, Unit 9). Peptides of the disclosure can also be synthesized by the well known solid phase peptide synthesis methods such as those described by Merrifield, J. Am. Chem. Soc., 85:2149, 1962; and Stewart and Young, Solid Phase Peptides Synthesis, Freeman, San Francisco, 1969, pp. 27-62, using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with a 1% acetic acid solution, which is then lyophilized to yield the crude material. The peptides can be purified by such techniques as gel filtration on Sephadex G-15 using 5% acetic acid as a solvent. Lyophilization of appropriate fractions of the column eluate yield homogeneous peptide, which can then be characterized by standard techniques such as amino acid analysis, thin layer chromatography, high performance liquid chromatography, ultraviolet absorption spectroscopy, molar rotation, or measuring solubility. If desired, the peptides can be quantitated by the solid phase Edman degradation.

The activity of the peptides of the disclosure can be determined using conventional methods known to those of skill in the art, such as in a “minimal inhibitory concentration (MIC)”, whereby the lowest concentration at which no change in OD is observed for a given period of time is recorded as the MIC. Alternatively, a “fractional inhibitory concentration (FIC)” assay can be used to measure synergy between the peptides of the disclosure, or the peptides in combination with known antibiotics. FICs can be performed by checkerboard titrations of peptides in one dimension of a microtiter plate, and of antibiotics in the other dimension, for example. The FIC is a function of the impact of one antibiotic on the MIC of the other and vice versa. A FIC of 1 indicates that the influence of the compounds is additive and a FIC of less than 1 indicates that the compounds act synergistically.

Peptides of the disclosure can be synthesized by commonly used methods such as those that include t-BOC or FMOC protection of alpha-amino groups. Both methods involve stepwise synthesis in which a single amino acid is added at each step starting from the C-terminus of the peptide (See, Coligan, et al., Current Protocols in Immunology, Wiley Interscience, 1991, Unit 9). Peptides of the disclosure can also be synthesized by the well known solid phase peptide synthesis methods such as those described by Merrifield, J. Am. Chem. Soc., 85:2149, 1962; and Stewart and Young, Solid Phase Peptides Synthesis, Freeman, San Francisco, 1969, pp. 27-62) using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with a 1% acetic acid solution, which is then lyophilized to yield the crude material. The peptides can be purified by such techniques as gel filtration on Sephadex G-15 using 5% acetic acid as a solvent. Lyophilization of appropriate fractions of the column eluate yield homogeneous peptide, which can then be characterized by standard techniques such as amino acid analysis, thin layer chromatography, high performance liquid chromatography, ultraviolet absorption spectroscopy, molar rotation, or measuring solubility. If desired, the peptides can be quantitated by the solid phase Edman degradation.

In one embodiment, a cathelicidin functional fragment polynucleotide/nucleic acid of the disclosure comprises a sequence of SEQ ID NO:5. In one aspect, a polynucleotide encoding a cathelicidin function fragment comprises SEQ ID NO:5 from about nucleotide 451 to 498, from about nucleotide 451 to 501, from about nucleotide 451 to 504, from about nucleotide 451 to 507, from about nucleotide 451 to 510, from about nucleotide 421 to 498, from about nucleotide 421 to 501, from about nucleotide 421 to 504, from about nucleotide 421 to 507, from about nucleotide 421 to 510, from about nucleotide 418 to 498, from about nucleotide 418 to 501, from about nucleotide 418 to 504, from about nucleotide 418 to 507, from about nucleotide 418 to 510, from about nucleotide 403 to 498, from about nucleotide 403 to 501, from about nucleotide 403 to 504, from about nucleotide 403 to 507, and from about nucleotide 403 to 510.

| (SEQ ID NO: 5) | |
|---|---|
| 1 | taaagcaaac cccagcccac accctggcag gcagccaggg atgggtggat caggaaggct |
| 61 | cctggttggg cttttgcatc aggctcaggc tgggcataaa ggaggctcct gtgggctaga |
| 121 | gggaggcaga catggggacc atgaagaccc aaagggatgg ccactccctg gggcggtggt |
| 181 | cactggtgct cctgctgctg ggcctggtga tgcctctggc catcattgcc caggtcctca |
| 241 | gctacaagga agctgtgctt cgtgctatag atggcatcaa ccagcggtcc tcggatgcta |
| 301 | acctctaccg cctcctggac ctggacccca ggcccacgat ggatggggac ccagacacgc |
| 361 | caaagcctgt gagcttcaca gtgaaggaga cagtgtgccc caggacgaca cagcagtcac |
| 421 | cagaggattg tgacttcaag aaggacgggc tggtgaagcg gtgtatgggg acagtgaccc |
| 481 | tcaaccaggc caggggctcc tttgacatca gttgtgataa ggataacaag agatttgccc |
| 541 | tgctgggtga tttcttccgg aaatctaaag agaagattgg caaagagttt aaaagaattg |
| 601 | tccagagaat caaggatttt ttgcggaatc ttgtacccag gacagagtcc tagtgtgtgc |
| 661 | cctaccctgg ctcaggcttc tgggctctga gaaataaact atgagagcaa tttcaaaaaa |
| 721 | aaaaaaaaaa aaaaaaaaa |

In another example, a polynucleotide encoding a cathelicidin functional fragment of the disclosure comprises a sequence of SEQ ID NO:7. In one aspect, a polynucleotide encoding a cathelicidin functional fragment comprises SEQ ID NO:7 from about nucleotide 460 to nucleotide 507.

| (SEQ ID NO: 7) | |
|---|---|
| 1 | atgcagttcc agagggacgt cccctccctg tggctgtggc ggtcactatc actgctgctg |
| 61 | ctactgggcc tggggttctc ccagaccccc agctacaggg atgctgtgct ccgagctgtg |
| 121 | gatgacttca accagcagtc cctagacacc aatctctacc gtctcctgga cctggatcct |
| 181 | gagccccaag gggacgagga tccagatact cccaagtctg tgaggttccg agtgaaggag |
| 241 | actgtatgtg gcaaggcaga gcggcagcta cctgagcaat gtgccttcaa ggaacagggg |
| 301 | gtggtgaagc agtgtatggg ggcagtcacc ctgaacccgg ccgctgattc ttttgacatc |
| 361 | agctgtaacg agcctggtgc acagcccttt cggttcaaga aaatttcccg gctggctgga |
| 421 | cttctccgca aaggtgggga gaagattggt gaaaagctta agaaaattgg ccagaaaatt |
| 481 | aagaattttt ttcagaaact tgtccctcag ccagagtag |

Such polynucleotides include naturally occurring, synthetic, and intentionally manipulated polynucleotides. For example, a cathelicidin functional fragment peptide or polynucleotide may be subjected to site-directed mutagenesis. A cathelicidin functional fragment polynucleotide includes sequences that are degenerate as a result of the genetic code. There are 20 natural amino acids, most of which are specified by more than one codon. Therefore, all degenerate nucleotide sequences are included so long as the amino acid sequence of a cathelicidin functional fragment peptide or variant encoded by the polynucleotide is functionally unchanged. Accordingly, a polynucleotide of the invention includes (i) a polynucleotide encoding a cathelicidin functional fragment; (ii) a polynucleotide encoding SEQ ID NO:1 or a variant thereof, or SEQ ID NO:2-4 or a variant thereof; (iii) a polynucleotide comprising SEQ ID NO:5 from about nucleotide 451 to 498, from about nucleotide 451 to 501, from about nucleotide 451 to 504, from about nucleotide 451 to 507, from about nucleotide 451 to 510, from about nucleotide 421 to 498, from about nucleotide 421 to 501, from about nucleotide 421 to 504, from about nucleotide 421 to 507, from about nucleotide 421 to 510, from about nucleotide 418 to 498, from about nucleotide 418 to 501, from about nucleotide 418 to 504, from about nucleotide 418 to 507, from about nucleotide 418 to 510, from about nucleotide 403 to 498, from about nucleotide 403 to 501, from about nucleotide 403 to 504, from about nucleotide 403 to 507, and/or from about nucleotide 403 to 510; (iv) a polynucleotide of (iii), wherein T is U; and (v) a polynucleotide comprising a sequence that is complementary to (iii) and (iv) above. It will be recognized that a cathelicidin functional fragment polynucleotide, may be operably linked to a second heterologous polynucleotide such as a promoter or a heterologous sequence encoding a desired peptide or polypeptide sequence.

The term “isolated” as used herein refers to a nucleic acid that is substantially free of proteins, lipids, and other nucleic acids with which an in vivo-produced nucleic acids naturally associated. Typically, the nucleic acid is at least 70%, 80%, 90% or more pure by weight, and conventional methods for synthesizing nucleic acids in vitro can be used in lieu of in vivo methods. As used herein, “nucleic acid” or “polynucleotide” refers to a polymer of deoxyribonucleotides or ribonucleotides, in the form of a separate fragment or as a component of a larger genetic construct (e.g., by operably linking a promoter to a nucleic acid encoding a peptide of the disclosure). Numerous genetic constructs (e.g., plasmids and other expression vectors) are known in the art and can be used to produce the peptides of the disclosure in cell-free systems or prokaryotic or eukaryotic (e.g., yeast, insect, or mammalian) cells. By taking into account the degeneracy of the genetic code, one of ordinary skill in the art can readily synthesize nucleic acids encoding the polypeptides of the disclosure. The nucleic acids of the disclosure can readily be used in conventional molecular biology methods to produce the peptides of the disclosure.

Polynucleotides encoding the cathelicidin functional fragments of the disclosure can be inserted into an “expression vector.” The term “expression vector” refers to a genetic construct such as a plasmid, infective agent or other vehicle known in the art that can be engineered to contain a polynucleotide encoding a peptide or polypeptide of the disclosure. Such expression vectors are typically plasmids that contain a promoter sequence that facilitates transcription of the inserted genetic sequence in a host cell. The expression vector typically contains an origin of replication, and a promoter, as well as genes that allow phenotypic selection of the transformed cells (e.g., an antibiotic resistance gene). Various promoters, including inducible and constitutive promoters, can be utilized in the disclosure. Typically, the expression vector contains a replicon site and control sequences that are derived from a species compatible with the host cell.

Transformation or transfection of a host cell with a polynucleotide of the disclosure can be carried out using conventional techniques well known to those skilled in the art. For example, where the host cell is E. coli, competent cells that are capable of DNA uptake can be prepared using the CaCl₂, MgCl₂ or RbCl methods known in the art. Alternatively, physical means, such as electroporation or microinjection can be used. Electroporation allows transfer of a polynucleotide into a cell by high voltage electric impulse. Additionally, polynucleotides can be introduced into host cells by protoplast fusion, using methods well known in the art. Suitable methods for transforming eukaryotic cells, such as electroporation and lipofection, also are known.

“Host cells” encompassed by of the disclosure are any cells in which the polynucleotides of the disclosure can be used to express the cathelicidin functional fragments of the disclosure. The term also includes any progeny of a host cell. Host cells, which are useful, include bacterial cells, fungal cells (e.g., yeast cells), plant cells and animal cells. For example, host cells can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology (1986)). As representative examples of appropriate hosts, there may be mentioned: fungal cells, such as yeast; insect cells such as Drosophila S2 and Spodoptera Sf9; animal cells such as CHO, COS or Bowes melanoma; plant cells, and the like. The selection of an appropriate host is deemed to be within the scope of those skilled in the art from the teachings herein.

Host cells can be eukaryotic host cells (e.g., mammalian cells). In one aspect, the host cells are mammalian production cells adapted to grow in cell culture. Examples of such cells commonly used in the industry are CHO, VERO, BHK, HeLa, CV1 (including Cos; Cos-7), MDCK, 293, 3T3, C127, myeloma cell lines (especially murine), PC12 and W138 cells. Chinese hamster ovary (CHO) cells are widely used for the production of several complex recombinant proteins, e.g. cytokines, clotting factors, and antibodies (Brasel et al., Blood 88:2004-2012, 1996; Kaufman et al., J. Biol Chem 263: 6352-6362, 1988; McKinnon et al., J Mol Endocrinol 6:231-239, 1991; Wood et al., J. Immunol. 145:3011-3016, 1990). The dihydrofolate reductase (DHFR)-deficient mutant cell lines (Urlaub et al., Proc Natl Acad Sci USA 77:4216-4220, 1980) are the CHO host cell lines commonly used because the efficient DHFR selectable and amplifiable gene expression system allows high level recombinant protein expression in these cells (Kaufman, Meth Enzymol 185:527-566, 1990). In addition, these cells are easy to manipulate as adherent or suspension cultures and exhibit relatively good genetic stability. CHO cells and recombinant proteins expressed in them have been extensively characterized and have been approved for use in clinical manufacturing by regulatory agencies.

Polynucleotides encoding the peptides of the disclosure can be isolated from a cell (e.g., a cultured cell), or they can be produced in vitro. A DNA sequence encoding a cathelicidin functional fragment of interest can be obtained by: 1) isolation of a double-stranded DNA sequence from genomic DNA; 2) chemical manufacture of a polynucleotide such that it encodes the cathelicidin functional fragment of interest; or 3) in vitro synthesis of a double-stranded DNA sequence by reverse transcription of mRNA isolated from a donor cell (i.e., to produce cDNA).

Included in the disclosure are polynucleotides that encode analogs, mutants, conservative variations, and variants of the peptides described herein. For example, an isolated polynucleotide encoding a cathelicidin functional fragment of the disclosure can comprise the sequence of SEQ ID NO:5 from about nucleotide 451 to 498, from about nucleotide 451 to 501, from about nucleotide 451 to 504, from about nucleotide 451 to 507, from about nucleotide 451 to 510, from about nucleotide 421 to 498, from about nucleotide 421 to 501, from about nucleotide 421 to 504, from about nucleotide 421 to 507, from about nucleotide 421 to 510, from about nucleotide 418 to 498, from about nucleotide 418 to 501, from about nucleotide 418 to 504, from about nucleotide 418 to 507, from about nucleotide 418 to 510, from about nucleotide 403 to 498, from about nucleotide 403 to 501, from about nucleotide 403 to 504, from about nucleotide 403 to 507, and from about nucleotide 403 to 510.

Any of various art-known methods for protein purification can be used to isolate the peptides of the disclosure. For example, preparative chromatographic separations and immunological separations (such as those employing monoclonal or polyclonal antibodies) can be used. Carrier peptides can facilitate isolation of fusion proteins that include the peptides of the disclosure. Purification tags can be operably linked to a cathelicidin functional fragment of the disclosure. For example, glutathione-S-transferase (GST) allows purification with a glutathione agarose affinity column. When either Protein A or the ZZ domain from Staphylococcus aureus is used as the tag, purification can be accomplished in a single step using an IgG-sepharose affinity column. The pOprF-peptide, which is the N-terminal half of the P. aeruginosa outer membrane protein F, can readily be purified because it is the prominent protein species in outer membrane preparations. If desired, the fusion peptides can be purified using reagents that are specifically reactive with (e.g., specifically bind) the cathelicidin functional fragment of the fusion peptide. For example, monoclonal or polyclonal antibodies that specifically bind the cathelicidin functional fragment can be used in conventional purification methods. Techniques for producing such antibodies are well known in the art.

In one aspect of the invention, vitamin D3 is contacted with a subject to promote/stimulate host cell defense mechanisms by, for example, stimulating cathlicidin production and processing to mount a more effective cell-mediated immune response. For example, as demonstrated in the specific examples below, vitamin D3 promotes cathelicidin activity in vitro and in vivo.

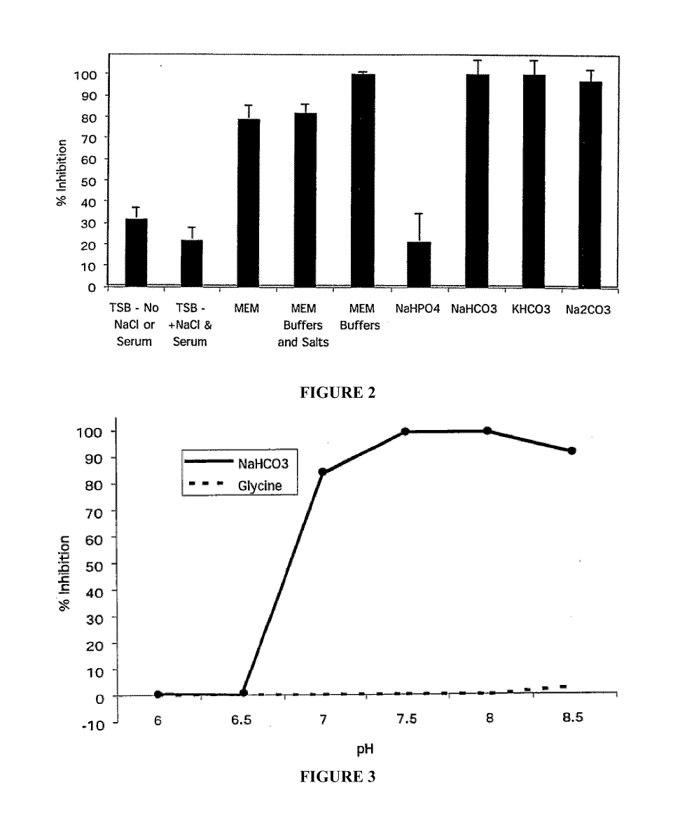

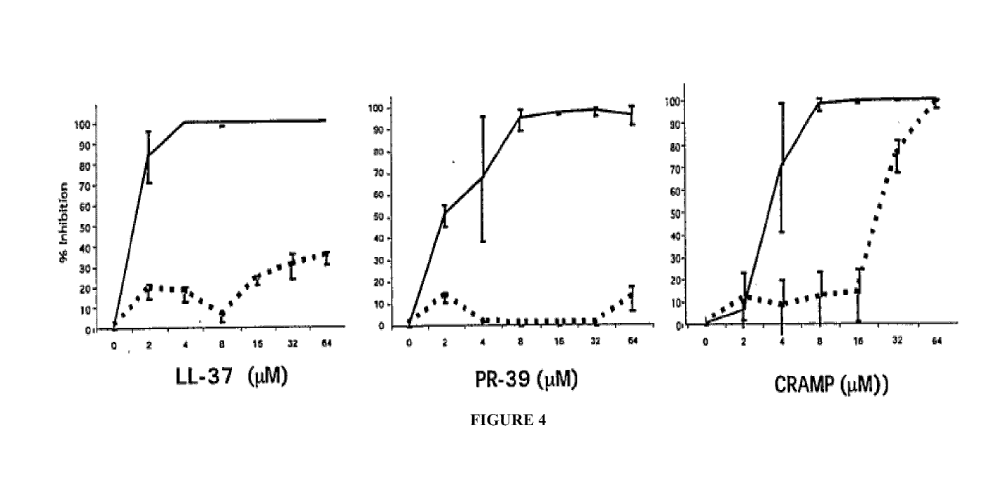

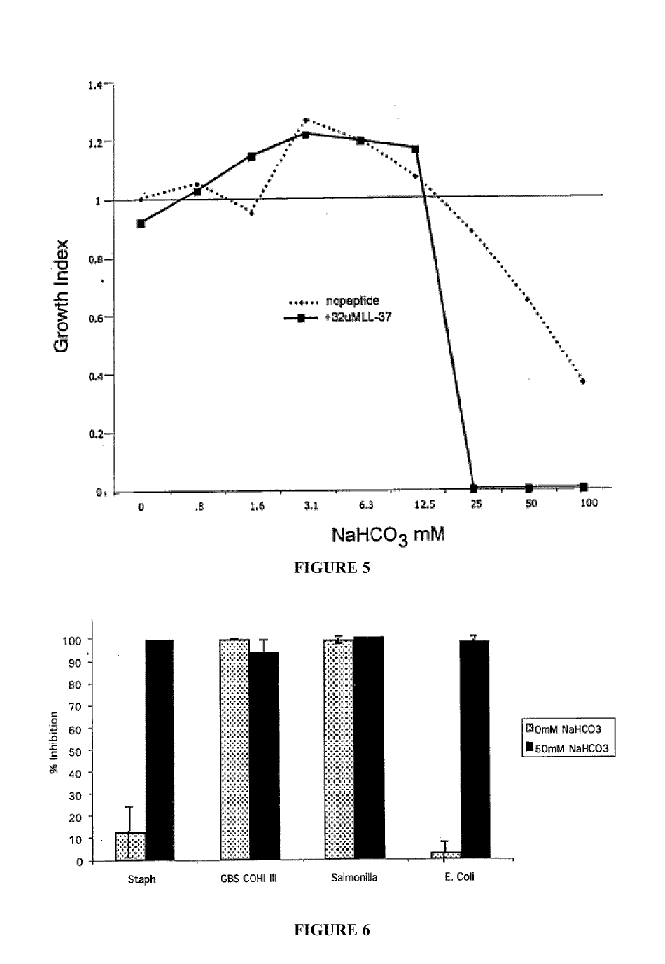

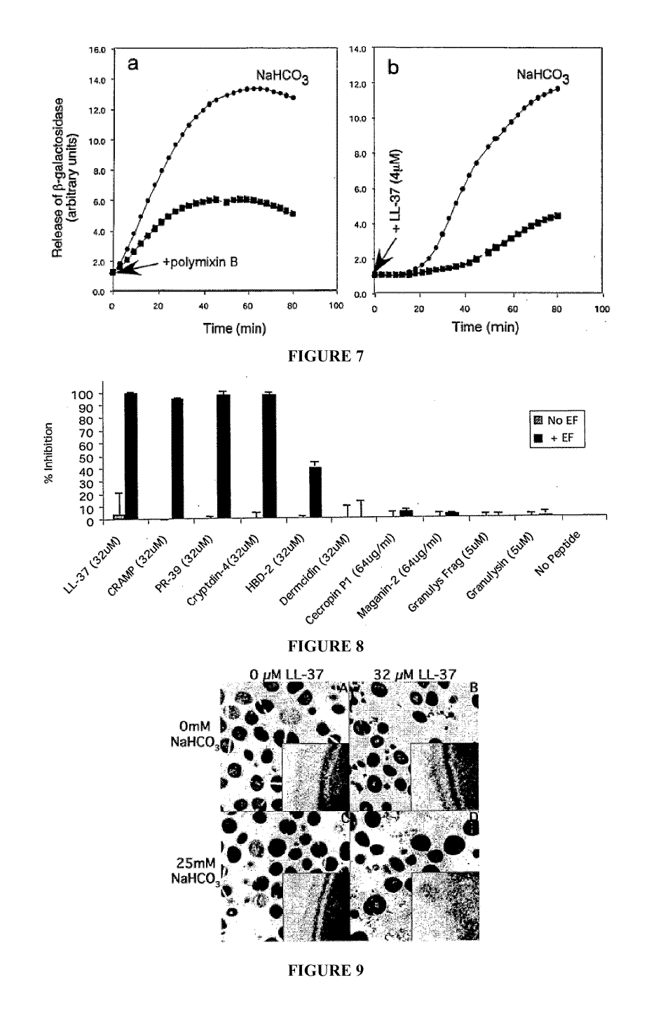

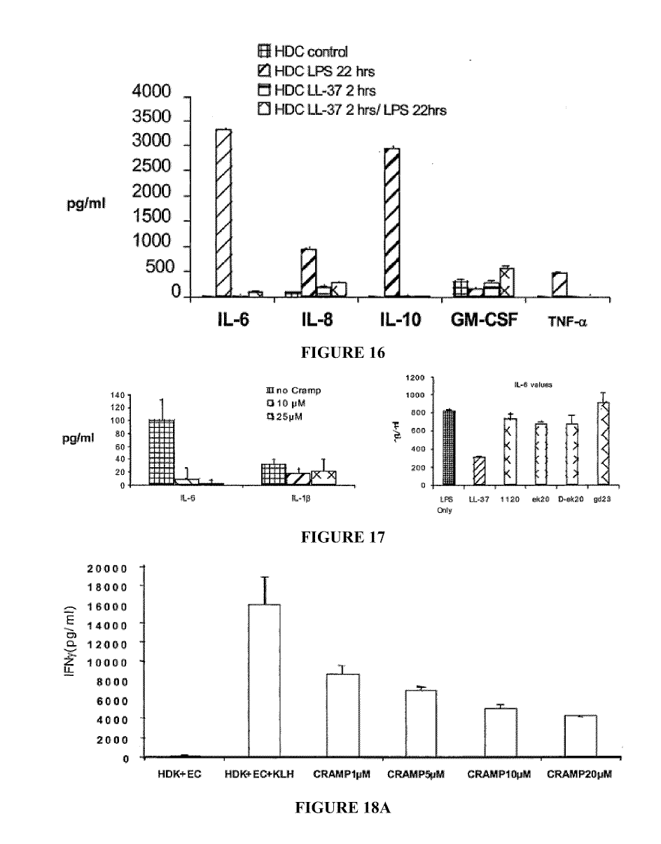

In another aspect of the invention, vitamin D3, carbonate and/or peptides of the disclosure are contacted with a subject to promote/stimulate host cell defence mechanisms by, for example, stimulating epithelia to release cytokines to mount a more effective cell-mediated immune response. For example, as demonstrated in FIGS. 1-9 bicarbonate promotes LL-37 activity. In addition, FIGS. 15-16 shows the effect of LL-37 and functional fragments on inflammatory cell recruitment and cytokine production.

The disclosure also provides a method for inhibiting the growth of a bacterium by contacting the bacterium with an inhibiting effective amount of a composition comprising vitamin D3 and a cathelicidin functional fragment of the disclosure. The term “contacting” refers to exposing the bacterium to the composition so that the peptide can inhibit, kill, or lyse bacteria. Contacting can occur in vivo, for example by administering the composition to a subject afflicted with a bacterial infection or susceptible to infection. In vivo contacting includes both parenteral as well as topical. “Inhibiting” or “inhibiting effective amount” refers to the amount of composition that is sufficient to cause, for example, a bacteriostatic or bactericidal effect or an anti-viral or an anti-fungal effect.

Bacteria that can be affected by the peptides of the disclosure include both gram-negative and gram-positive bacteria. For example, bacteria that can be affected include Staphylococcus aureus, Streptococcus pyogenes (group A), Streptococcus sp. (viridans group), Streptococcus agalactiae (group B), S. bovis, Streptococcus (anaerobic species), Streptococcus pneumoniae, and Enterococcus sp.; Gram-negative cocci such as, for example, Neisseria gonorrhoeae, Neisseria meningitidis, and Branhamella catarrhalis; Gram-positive bacilli such as Bacillus anthracis, Bacillus subtilis, P.acne Corynebacterium diphtheriae and Corynebacterium species which are diptheroids (aerobic and anerobic), Listeria monocytogenes, Clostridium tetani, Clostridium difficile, Escherichia coli, Enterobacter species, Proteus mirablis and other sp., Pseudomonas aeruginosa, Klebsiella pneumoniae, Salmonella, Shigella, Serratia, and Campylobacter jejuni. Infection with one or more of these bacteria can result in diseases such as bacteremia, pneumonia, meningitis, osteomyelitis, endocarditis, sinusitis, arthritis, urinary tract infections, tetanus, gangrene, colitis, acute gastroenteritis, impetigo, acne, acne posacue, wound infections, born infections, fascitis, bronchitis, and a variety of abscesses, nosocomial infections, and opportunistic infections. The method for inhibiting the growth of bacteria can also include contacting the bacterium with the peptide in combination with one or more antibiotics.

Fungal organisms may also be affected by the compositions of the disclosure and include dermatophytes (e.g., Microsporum canis and other Microsporum sp.; and Trichophyton sp. such as T. rubrum, and T. mentagrophytes), yeasts (e.g., Candida albicans, C. Tropicalis, or other Candida species), Saccharomyces cerevisiae, Torulopsis glabrata, Epidermophyton floccosum, Malassezia furfur (Pityropsporon orbiculare, or P. ovale), Cryptococcus neoformans, Aspergillus fumigatus, Aspergillus nidulans, and other Aspergillus sp., Zygomycetes (e.g., Rhizopus, Mucor), Paracoccidioides brasiliensis, Blastomyces dermatitides, Histoplasma capsulatum, Coccidioides immitis, and Sporothrix schenckii.

A composition(s) of the disclosure can be administered to any host, including a human or non-human animal, in an amount effective to inhibit growth of a bacterium, infective agent, and/or fungus. Thus, the peptides are useful as antimicrobial agents, antiviral agents, and/or antifungal agents.

Any of a variety of art-known methods can be used to administer a composition (e.g., a vitamin D3 containing composition or a vitamin D3+peptide composition) to a subject. For example, the composition of the disclosure can be administered parenterally by injection or by gradual infusion over time. The composition can be administered intravenously, intraperitoneally, intramuscularly, subcutaneously, intracavity, or transdermally.

In another aspect, a composition of the disclosure may be formulated for topical administration (e.g., as a lotion, cream, spray, gel, or ointment). Such topical formulations are useful in treating or inhibiting microbial, fungal, and/or viral presence or infections on the eye, skin, and mucous membranes such as mouth, vagina and the like. Examples of formulations in the market place include topical lotions, creams, soaps, wipes, and the like. It may be formulated into liposomes to reduce toxicity or increase bioavailability. Other methods for delivery of the composition include oral methods that entail encapsulation of the vitamin D3 or vitamin D3+peptide in microspheres or proteinoids, aerosol delivery (e.g., to the lungs), or transdermal delivery (e.g., by iontophoresis or transdermal electroporation). Other methods of administration will be known to those skilled in the art.

Preparations for parenteral administration of a composition of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

The disclosure provides a method for inhibiting a topical bacterial, viral and/or fungal-associated disorder by contacting or administering a therapeutically effective amount of a composition of the disclosure to a subject who has, or is at risk of having, such a disorder. The term “inhibiting” means preventing or ameliorating a sign or symptoms of a disorder (e.g., a rash, sore, and the like). Examples of disease signs that can be ameliorated include an increase in a subject's blood level of TNF, fever, hypotension, neutropenia, leukopenia, thrombocytopenia, disseminated intravascular coagulation, adult respiratory distress syndrome, shock, and organ failure. Examples of subjects who can be treated in the disclosure include those at risk for, or those suffering from, a toxemia, such as endotoxemia resulting from a gram-negative bacterial infection, venom poisoning, or hepatic failure. Other examples include subjects having a dermatitis as well as those having skin infections or injuries subject to infection with gram-positive or gram-negative bacteria, a infective agent, or a fungus. Examples of candidate subjects include those suffering from infection by E. coli, Hemophilus influenza B, Neisseria meningitides, staphylococci, or pneumococci. Other patients include those suffering from gunshot wounds, renal or hepatic failure, trauma, burns, immunocompromising infections (e.g., HIV infections), hematopoietic neoplasias, multiple myeloma, Castleman's disease or cardiac myxoma. Those skilled in the art of medicine can readily employ conventional criteria to identify appropriate subjects for treatment in accordance with the disclosure.

Generally, the optimal dosage of the composition will depend upon the disorder and factors such as the weight of the subject, the type of bacteria, infective agent or fungal infection, the weight, sex, and degree of symptoms. Nonetheless, suitable dosages can readily be determined by one skilled in the art.

If desired, a suitable therapy regime can combine administration of a vitamin D3 composition of the disclosure with one or more additional therapeutic agents (e.g., an inhibitor of TNF, an antibiotic, and the like). The composition(s), other therapeutic agents, and/or antibiotic(s) can be administered, simultaneously, but may also be administered sequentially. Suitable antibiotics include aminoglycosides (e.g., gentamicin), beta-lactams (e.g., penicillins and cephalosporins), quinolones (e.g., ciprofloxacin), and novobiocin.

The vitamin D3 compositions of the disclosure can be used to treat damaged tissue, such as wounds (in particular chronic wounds), more effectively. As demonstrated herein, cathelicidin functional fragments of the disclosure are effective protease inhibitors. The cathelicidin functional fragments of the disclosure are capable of inhibiting the action of specific proteins that are upregulated in a wound environment wherein those proteins have an adverse effect in the wound environment. Typically the adverse effect is a deleterious effect on wound healing. Hence, the cathelicidin functional fragments of the disclosure can be used to inhibit the deleterious effects of proteases that are upregulated in a wound environment. Accordingly, the administration or vitamin D3 upregulates cathelicidins thereby inhibiting protease damage. Simultaneously, the cathelicidin functional fragments of the disclosure also are capable of serving as antibacterial agents that reduce the risk of infection at a wound site by inhibiting proteases produced by pathogens in the wound site.

A vitamin D3 compositions of the invention (e.g., comprising a cathelicidin functional fragment) can be administered to any host, including a human or non-human animal, in an amount effective to inhibit growth of a infective agent, bacterial or fungus.

The following examples are intended to illustrate but not limit the disclosure. While they are typical of those that might be used, other procedures known to those skilled in the art may alternatively be used.

### EXAMPLES

### Materials and Methods

Sweat collection and processing. Sweat was collected on paper tissues (Kimwipes, Kimberly-Clark, Ga.) from healthy volunteers after exercise. After collection, 20 ml of sweat was centrifuged at 2000 g for 15 min. at 4° C., filtered through a 0.20 ml filter (Acrodisc syringe filter, 0.2 mm, low protein binding, Fisher scientific, Tustin, Calif.), and frozen at −80° C. For some experiments sweat was lyophilized to dryness, then suspended in 400 μl of distilled water (DW: cell culture grade, endotoxin free, GibcoBRL, Grand island, NY). For analysis of LL37 processing by sweat, 1.6 nmol of LL-37 synthetic peptide was incubated in 50 ml of sweat for 0, 1, 6, 24 hrs at 37° C. or 4° C. In some experiments proteinase inhibitors including; mixed protease inhibitor cocktail (1tab/10 ml, Roche, Indianapolis, Ind.), 100 mg/ml Bestatin, 10 mg/ml E-64, 10 mg/ml Aprotinin, (Sigma, St Louis, Mo.); 100 mM AEBSF, 100 mM Neutrophil elastase inhibitor (NEI) or 100 mM leukocyte elastase inhibitor (LEI) (Calbiochem, San Diego, Calif.) were added during incubation. After incubation, 2 ml was assayed by radial diffusion assay to determine antibacterial activity. For analysis by HPLC 32 nmoles LL-37 was incubated in 100u1. To control for potential contamination from paper tissues, parallel processing was done on tissues soaked in phosphate-buffered saline (PBS:137 mM NaCl, 2.7 mM KCL, 4.3 mM Na2HPO4-H2O, 1.4 mM KH2PO4, pH 7.4). No antimicrobial activity was detectable in these preparations. Protein concentrations were evaluated by BCA assay (protein assay reagent, Pierce, Rockford, Ill.) or Bradford protein assay (Bio-Rad, Hercules, Calif.) according to manufacture's instructions. Human tissue and blood collection was approved by the UCSD Human Research Protection Program.

Peptide synthesis. Dermcidin, LL-37, RK-31, KS-30, and KR-20 peptides were commercially prepared by Synpep Corporation, Dublin, Oreg. Peptide amino acid sequences were LLGDFFRKSKEKIGKEFKRIVQRIKDFLRNLVPRTES (LL-37), RKSKEKIGKEFKRIVQRIKDFLRNLVPRTES (RK-31), KSKEKIGKEFKRIVQRIKDFLRNLVPRTES (KS-30), KRIVQRIKDFLRNLVPRTES (KR-20), SSLLEKGLDGAKKAVGGLGKLGKDAVEDLESVGKGAVHDVKDVLDSV dermcidin). All 5 synthetic peptides were purified by HPLC and identity confirmed by mass spectrometry.

HPLC chromatography. Peptide separation was performed using an AKTA purification system (Amersham Pharmacia Biotech, Piscataway, N.J.) on a Sephasil peptide C18 column (12 mm, ST 4.6/250, Amersham Pharmacia Biotech, Piscataway, N.J.). Concentrated human sweat, or LL-37 incubated in sweat, was separated by reversed phase (RP-HPLC) following column equilibration in 0.1% TFA at a flow rate of 0.5 ml/ml and eluted using gradients of 0-35% and 35-60% acetonitrile for 16 min, 67 min, or 0-60% acetonitrile for 120 min. Column effluent was monitored at 214, 230, and 280 nm. All collected fractions (1 ml) were lyophilized and suspended in 10 ml of DW for antimicrobial radial diffusion assay.

Western and Immunoblot analysis. Fractions purified by HPLC as described above were evaluated by quantitative dot-blot and Western Blot. 2 μl of each fraction was compared to a standard curve of synthetic LL37 peptide applied onto PVDF membrane (Immobilon-P, Millipore, Mass.). Antibody used was rabbit anti-LL-37 polyclonal antibody derived and affinity purified against the entire LL-37 peptide. For immunoblot, membranes were blocked (0.1% TTBS: 5% nonfat milk in 0.1% Tween 20/tris buffered saline (TBS: 150 mM NaCl, 10 mM Tris Base, pH 7.4)) for 60 min at room temperature, and then rabbit anti LL37 polyclonal antibody (1:5000 in blocking solution) was incubated with the membrane overnight at 4° C. After washing 3× with 0.1% TTBS, horseradish peroxidase labeled goat anti rabbit:antibody (1:5000 in the blocking solution, (DAKO, CA) was incubated with the membrane for 60 min, room temperature. After washing the membrane again with 0.1% TTBS, the membrane was immersed in ECL solution (Western Lightning Chemiluminescence Reagents Plus, New Lifescience Products, Boston, Mass.) for 60 sec then exposed to X-ray film (Kodak). For, Western-blot analysis, sweat samples (10 μl) were separated by 16.5% tristricine/peptide gel (BIO-RAD, Hercules, Calif.), and then transferred on to a PVDF membrane (Immobilon-P, Millipore, Mass.). For positive control, 5 μmol LL-37 synthetic peptide was applied.

Mass spectrometry and Protein sequence analysis. Mass spectrometry was performed by Center for Mass Spectrometry, The Scripps Research Institute (La Jolla, Calif.). MALDI-MS spectra were obtained with a Voyager DE-RP MALDI-TOF mass spectrometer (PerSeptive Biosystems, Framingham, Mass.) equipped with a nitrogen laser (337 nm, 3-ns pulse). Spectra were collected in reflector mode. The accelerating voltage in the ion source was 20 kV. Data were acquired with a transient recorder with 2-ns resolution. The matrix used in this work was -cyano-4-hydroxycinnamic acid dissolved in water/acetonitrile (1:1, v/v) to give a saturated solution at room temperature. To prepare the sample for analysis, 1 μl of the peptide solution (containing 1-10 pmol of protein in 0.1% trifluoroacetic acid) was added to 1 μl of the matrix solution and applied to a stainless steel sample plate. The mixture was then allowed to air dry on the sample plate before being introduced into the mass spectrometer. Each spectrum was produced by accumulating data using 128 laser pulses. Mass assignments were assigned with an accuracy of approximately ±0.1% (±1 Da/1000 Da). Protein sequence analysis for target HPLC fractions was performed by Division of Biology Protein Sequencer Facility, University of California, San Diego.

The amino acid sequencing was performed on an Applied Biosystems Procise Model 494 sequencer using the “pulsed-liquid” program supplied by the manufacture.

Antimicrobial assays. For screening of antimicrobial activity of HPLC fractions, radial diffusion assay was used. Lyophilized HPLC fractions were dissolved in DW (GibcoBRL, Grand Island, N.Y.), and tested against Staphylococcus aureus mprF. Thin plates (1 mm) of 1% agarose in 0.5% tryptone containing approximately 1×10⁶ cells/ml of S. aureus mprF were used. 1 mm wells were punched in the plates and 2 ml of samples dissolved in tissue culture grade sterile water were loaded in each well.

As a positive control, synthetic LL-37 was applied to separate wells. After incubation at 37° C. overnight, the inhibition zones were recorded by CCD camera and diameters measured. To evaluate antimicrobial activity against wild-type S. aureus (clinical isolate), and enteroinvasive E. coli 029, both radial diffusion and solution killing assays were done. Radial diffusion assays were done as described for S. aureus mprF. Solution killing was done in 10% TSB in 10 mM PB (TSB=30 gm/L Tryptic Soy Broth, Sigma, St Louis Mo.; 20×PB=27.6 g/L NaH2PO4-H2O, 53.65 g/L Na2HPO4-7H2O, pH 7.4). Bacteria in log-phase growth were suspended to 1×10⁶ cells/ml, peptide added and incubated at 37° C. for 2 hr. Bacteria were then plated on TSB agar (TSB, Bactoagar 13 g/L, Becton Dickinson, Sparks Md.) for direct colony count and determination of CFU.

Activity against Group A Streptococcus (NZ131) was done only in solution assay as described. To evaluate antimicrobial activity in high salt conditions, solution assay was done in 10% TSB/10 mM PBS with several salt concentrations (concentration of NaCl; 10, 50, 100, 150, 300, 500 mM). Action against the C. albicans was determined in Dixon medium (0.6% Peptone, 4% Malt Extract, 1% Glucose, 0.1% Ox Bile, 1% Tween-80) in sterile 96-well microtiter plates (Corning Inc., Corning, N.Y.) at a final volume of 50 ml. The assay mixtures contained 1-2.5×10⁴ PFU/ml freshly grown Candida, 20% Dixon medium, 0.6 mM phosphate buffer pH 7, and 16 mg/ml chloramphenicol. Microtiter plates were incubated at 37° C. for 24 h with peptides, then plated on Dixon agar to determine the MFC (minimum fungicide concentration).

Hemolysis Assay. Hemolytic activity was determined on human whole blood. Freshly obtained whole blood was washed 3× in PBS and resuspended in PBS at its original volume containing peptides at indicated concentrations. Samples were incubated at 37° C. for 1.5 to 3 h, and hemolysis determined by centrifuging at 300 g and measurement of the absorbance of the supernatant at 578 nm. Hemolytic activity of each peptide was expressed as the percentage of total hemoglobin released compared to that released by incubating with 0.1% Triton X-100.

Measurement of IL-8 release from keratinocytes Normal human keratinocytes (NHK) were cultured in EpiLife cell medium (Cascade Biologics, Portland, Oreg.) containing 0.06 mM Ca2+, 1× EpiLife-defined growth supplement (EDGS), 50 U/ml penicillin and 50 mg/ml streptomycin (Invitrogen, Carlsbad, Calif.). Keratinocytes were seeded in a 96-well plate and grown to confluence under standard tissue culture conditions. Cells were incubated with 3 mM or 10 mM LL-37 or LL37-derived peptides (e.g., cathelicidin functional fragments) for 8 hours at 37° C. Supernatants were collected and stored at −20° C. overnight. IL-8 ELISA Assay was performed according to the manufacturer's instructions (BD OptEIA, Pharmingen, San Diego, Calif.). Supernatants were diluted 1:10 for assay. Simultaneously, LDH assays (Roche, Indianapolis, Ind.) were used to assess the cytotoxicity of peptides to keratinocytes. LDH release following peptide exposure was compared to release induced by 1% Triton X100.

### Results

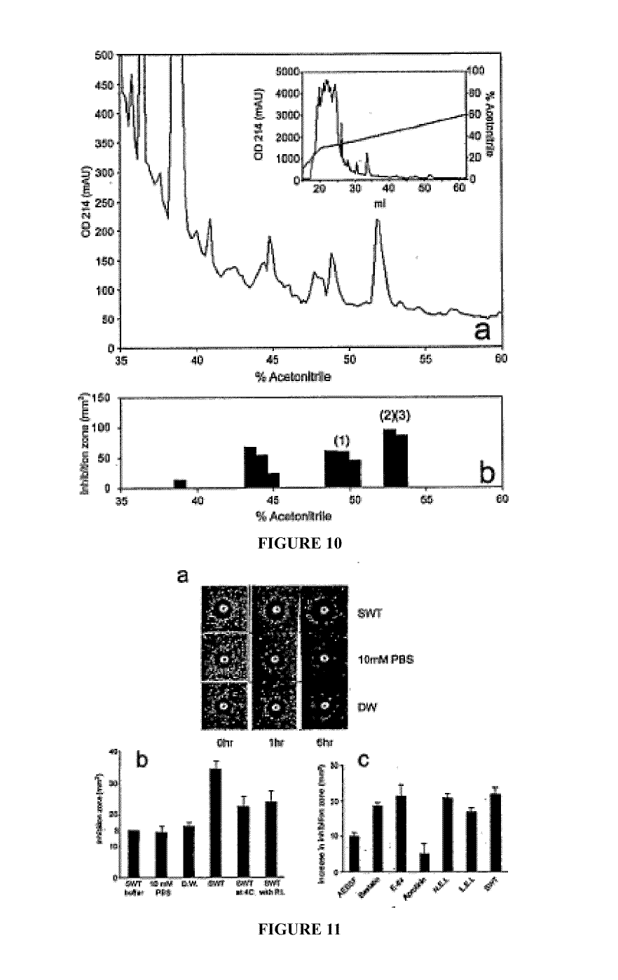

To identify antimicrobial activity present at the skin surface, human sweat was collected from normal volunteers, concentrated, and separated by high performance liquid chromatography (HPLC). Fractions of the material eluted between 35-65% acetronitrile were individually evaluated for the ability to inhibit the growth of S. aureus mprF by radial diffusion assay (FIG. 10). Multiple distinct fractions were found to be active in this assay. Prior evaluations of human sweat have shown that the antimicrobial peptides LL-37 and dermcidin are produced by the eccrine apparatus and secreted into the topical soluble environment of sweat. As expected, these molecules were detectable in the sweat preparation shown in FIG. 10. The presence of LL-37 was confirmed by immunoblot analysis with antibody specific to LL-37 and by MALDI-TOF mass spectrometry. The dermcidin peptides, DCD and DCD-1L, were identified by MALDI-TOF mass spectrometry and N-terminal amino acid sequencing.

Immunoblot analysis with anti-LL-37 antibody of all fractions isolated by HPLC from the human sweat suggested that other molecules related to LL-37 may be present in fractions eluting between 43 and 48% acetonitrile. These fractions were associated with antimicrobial activity but not identifiable by MALDI-TOF mass spectrometry and N-terminal amino acid sequencing from the concentrated sweat preparations. Based on the immunoreactivity and elution profile it was hypothesized that these antimicrobial molecules were alternative forms of LL-37 that were further processed in sweat to unique cathelicidin peptides. To test this, human sweat was freshly collected, sterilely filtered, then synthetic LL-37 added to a final concentration of 32 mM. The relative ability of this solution to inhibit the growth of S. aureus mprF was then evaluated. Following incubation at 37° C. in sweat, LL-37 increased the zone of inhibition and apparent antimicrobial activity (FIG. 11 panel a). Incubation of LL-37 under identical conditions in PBS or distilled water did not affect activity. This increase in apparent antimicrobial activity did not occur at 4° C. and was inhibited by the addition of protease inhibitor cocktail, thus suggesting the gain in antimicrobial function was a consequence of an enzymatic process (FIG. 11 panel b). Addition of specific inhibitors of potential processing enzymes demonstrated that the serine protease inhibitors AEBSF and Aprotinin were most effective in blocking the increase in antimicrobial activity generated by incubation of LL-37 in sweat (FIG. 11 panel c).

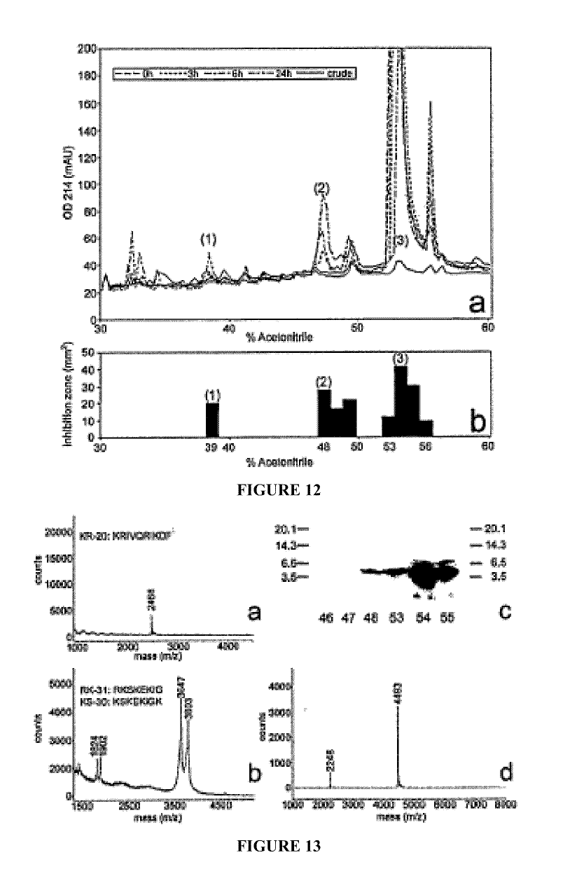

An increase in the relative ability of LL-37 to inhibit growth of S. aureus mprF suggested that the enzymatic processing of this peptide resulted in alternative forms with either increased direct antimicrobial activity or an ability to synergize with the parent peptide. To identify these processed forms of LL-37, synthetic peptide was incubated with the sterile sweat preparation and separated by HPLC after various periods of incubation (FIG. 12 panel a). An increase in the relative abundance of several proteins was seen over time.

Correlation of this profile with antimicrobial activity showed fractions eluding at 39% and 48% acetonitrile gained antimicrobial activity coincident with and increase in relative abundance of peptides eluting at these positions (FIG. 12 panel b). A third major peak of antimicrobial activity seen between 53 and 56% acetonitrile showed a relative decrease in abundance as estimated by absorbance at 214 nm. MALDI-TOF mass spectrometry and N-terminal sequencing of the peptide eluding at 39% acetonitrile identified this as a 20 amino acid cathelicidin derivative, KR-20 (FIG. 13 panel a). Similar analysis of peptides eluding at 48% acetonitrile identified two additional cathelicidin peptides RK-31 and KS-30 (FIG. 13 panel b). Western blot analysis with antibody against LL-37 confirmed antigenic similarity of RK-31 and KS-30 to parent LL-37 (FIG. 13 panel c). KR-20 was not detectable with this antibody. Material eluting at 55-56% acetonitrile and decreasing in abundance with incubation was identified as LL-37 by both Western blot and Mass spec analysis (FIG. 13 panels c and d).

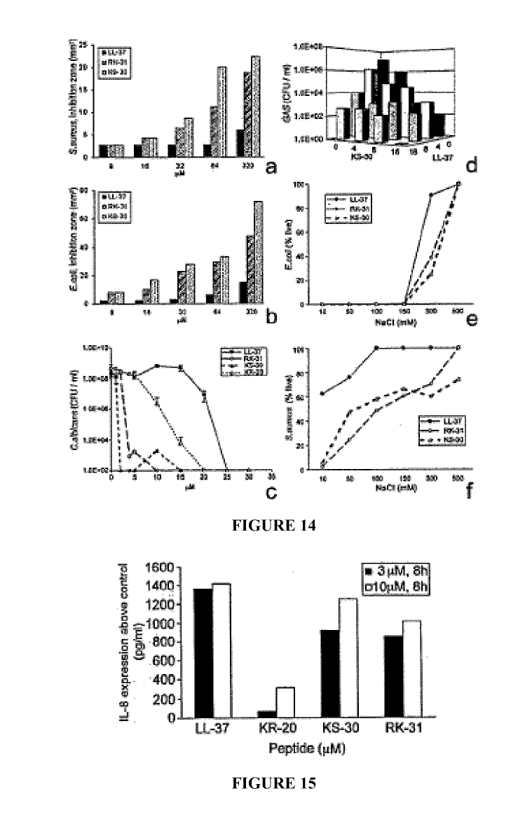

The newly described human cathelicidin peptides KR-20, RK-31 and KS-30 eluded at positions corresponding to unidentified antimicrobial activity seen in crude sweat preparations partially purified in FIG. 10. The low relative abundance of these peptides, yet easily detectable antimicrobial activity suggested that these peptides might gain antimicrobial activity with processing when compared to LL-37. To compare the antimicrobial activity of these cathelicidins, purified synthetic peptides corresponding to LL-37, RK-31, KS-30 and KR-20 were assessed by both radial diffusion assay and solution assay against a variety of microbes (FIG. 14). RK-31 and KS-30 showed greatly increased action against wild-type S. aureus and E. coli. And all three new peptides showed increased fungicidal activity against Candida albicans. Furthermore, these peptides were synergistic, killing bacteria at lower concentrations when present together, (FIG. 14 panel d), and maintained activity at increased salt conditions (FIG. 14 panels e and f). Hemolytic activity against human erythrocytes was minimal as seen by assay at a concentration 5 to 10 times greater than that required for antimicrobial activity (Table 2).

TABLE 2
Antimicrobial and hemolytic activity of human sweat peptides.

| dial Diffusion Assay (μm) | | | | quid Assay (μm) | | |
|---|---|---|---|---|---|---|
| | aureus | | coli | | albicans | |
| | cF | aureus (wt | 29) | S (NZ131) | CC 14053 | hemolysis |
| −37 | | 4 | | | | |
| −31 | | | | | | |
| −30 | | | | | | 3 |
| −20 | | 4 | 4 | | | |
| D | 4 | 4 | 4 | 4 | 7 | |

Radial diffusion assay results represent minimal inhibitory concentrations (MIC) determined for peptides against indicated bacteria. Liquid assay results represent minimal concentration required to kill Group A Streptococcus (GAS) or Candida albicans. Percent hemolysis is shown for peptides at the following concentrations: LL-37 and KR-20 = 100 μM; RK-31 = 44 μM; KS-30 = 88 μM. nd = not done.

However, despite gaining antimicrobial activity by processing to shorter forms of the cathelicidin peptide, hemolytic activity of these antimicrobials decreased relative to LL-37.

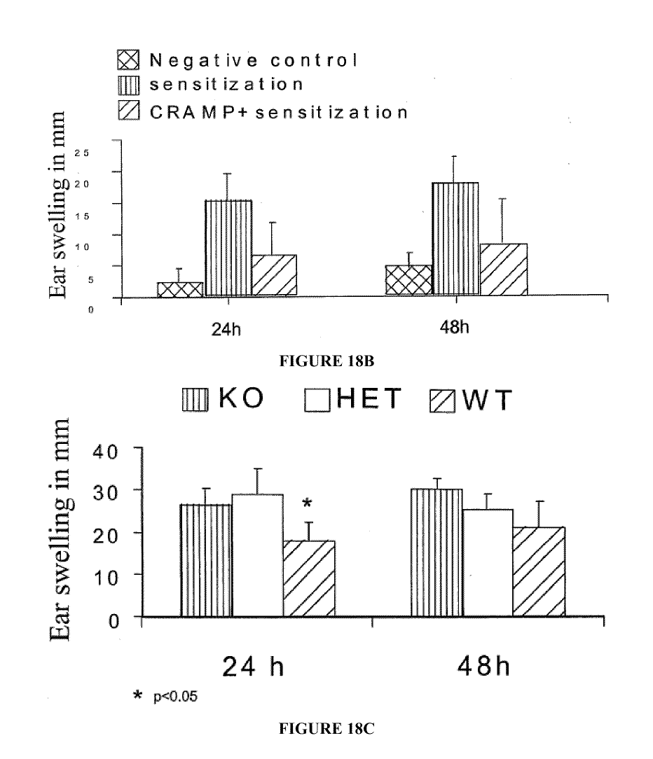

LL-37 was demonstrated to bind FPRL-1, induces neutrophil and mast cell chemotaxis (see, e.g., FIG. 16). LL-37 blocks pro-inflammatory chemokine release from dendritic cells, but induces pro-inflammatory release from keratinocytes. CRAMP has similar effect in mouse dendritic cells (FIG. 17) and inhibits antigen presentation in vitro (FIG. 18A-C). This effect is dissociated from cathelicidin functional fragments.

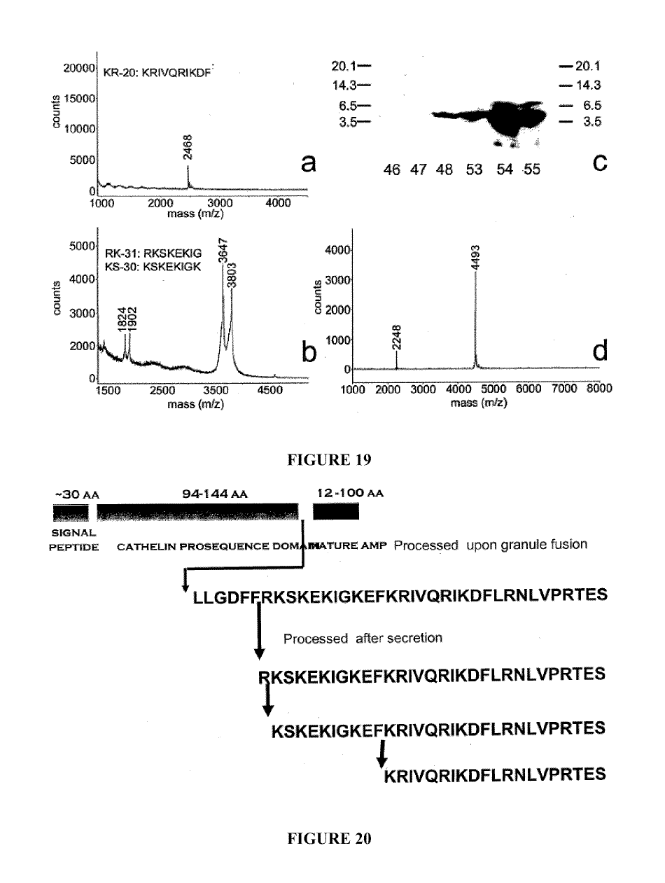

FIG. 19A-C shows the identification of cathelicidin function fragments on the normal skin surface by HPLC. FIG. 20 shows a schematic of cathelicidin processing on the normal skin surface.

Patients and treatments. Human skin wounds were collected following informed consent by inducing 5 mm full-thickness wounds in volunteers (n=5) under aseptic conditions with local anesthesia as described. At 24 hours after incision, 4 mm punch biopsies were taken from the site of injury and immediately fixed in formalin for immunostaining or frozen in liquid N2 for subsequent RNA isolation. Intact skin from healthy volunteers served as control (n=4). To examine the effects of topical 1,25D3, healthy skin of volunteers (n=7) was treated once topically with 1,25D3 (1.0 mM) and left under occlusion for 4 days. Vehicle treated contralateral skin served as a control. After treatment, skin biopsies were obtained from both sites, fixed in formalin 10% and embedded in paraffin for immunostaining. All treatments and sample acquisitions, including skin biopsies, were approved by the committees on investigations involving human subjects.

Animal studies. Skin wounds were collected from CYP27B1 (−/−) mice or their wildtype littermates (n=5 per group) 24 hours after induction of a 5 mm full-thickness wound under aseptic conditions. Non-injured skin from the same animal served as control. Skin samples were immediately frozen for subsequent RNA isolation.

Cell culture and stimuli. Human keratinocytes were grown in serum-free EpiLife® cell culture media (Cascade Biologics; Portland, Oreg.) containing 0.06 mM Ca2+ and 1× EpiLife® defined growth supplement (EDGS) at 37° C. under standard tissue culture conditions. Stock cultures were maintained for up to five passages in this media with the addition of 50 U/mL penicillin and 50 μg/mL streptomycin. HaCaT keratinocytes were cultured in DMEM (Sigma, St. Louis, Mo.) with 10% FCS. Cells at 50-70% confluence were stimulated in fresh medium after preincubation with 1,25(OH)₂ vitamin D3 (1,25D3; 0.1-100 nM, 4-24 h; Sigma). Alternatively, NHEK were stimulated in the presence of 1,25D3 (0.1-100 nM). Stimuli included TLR2,6 ligands Malp-2 (0.001-0.5 μg/ml; Alexis Biochemicals, Carlsbad, Calif.), FSL-1 (1-2 μg/ml, Invivogen, San Diego, Calif.) and zymosan (50 μg/ml; Invivogen), as well as flagellin (50 ng/ml; Alexis), LPS (1 μg/ml; Sigma), lipoteichoic acid (LTA; 10 μg/ml; Sigma), 19 kDA lipopeptide (10 μg/ml, a kind gift from S. Krutzik, Division of Dermatology, UCLA), CpG DNA (10 μg/ml; Sigma), Ca2+ (1.7 mM), LPS (1 μg/ml; Sigma), TNFα (20 ng/ml; Chemicon Temecula, Calif.) and TGFβ1 (0.1-1 μg/ml; R&D Systems, Minneapolis, Minn.). A TLR2 blocking antibody (αTLR2; clone TL2.1, 5 μg/ml, eBioscience, San Diego, Calif.) was utilized to investigate the role of TLR2 signaling. Furthermore, keratinocytes were pretreated with a vitamin D receptor antagonist (VDR-antagonist ZK159222 Schering AG; 10-7M) or the CYP inhibitor itraconazole (ITRA; 10-7M) before stimulation with TGFβ in the presence of 25OH vitamin D3 (25D3, 10 nM, Sigma).

Organotypic culture. Epiderm™ human skin-like tissue constructs were obtained from MatTek (Ashland, Mass.) and cultured according to manufacturer's instructions. Skin constructs were cultured in maintenance media (EPI-100-MM, MatTek) and stimulated with Malp-2 (0.1 μg/ml) for 24 hours in the presence of 1,25D3 (100 nM). Skin constructs were cut after stimulation and transferred to Trizol® (Invitrogen, Carlsbad, Calif.) for RNA extraction or immediately frozen in Tissue-Tek® (Sakura, Torrance, Calif.) for immunostaining.

Real-time RT-PCR (qPCR). RNA was extracted from cells, epidermal constructs or tissue using Trizol® and 1 μg RNA was reverse transcribed using iScript® (BioRad, Hercules, Calif.). Expression of cathelicidin and GAPDH was analysed by real-time RT-PCR. Predeveloped Taqman® assay probes (ABI, Foster City, Calif.) were used for the analyses of the expression of CYP24A1 (24-hydroxylase), CYP27B1 (1α-hydroxylase), CD14, CD36, TLR1, TLR2, TLR4, TLR6 and murine CD14. All analyses were performed in triplicate from 2-5 independent experiments in an ABI Prism® 7000 Sequence Detection System. Fold induction relative to the vehicle treated control in in vitro experiments was calculated using the comparative Ct method, where ΔΔCt is ΔCt(stimulant)-ΔCt(vehicle), ΔCt is Ct(gene)-Ct(GAPDH) and Ct is the cycle at which an arbitrary detection threshold is crossed. For quantification of transcript abundance in tissue samples target gene expression was normalized to GAPDH and compared to non-treated or non-injured skin.

Promoter analysis and site directed mutagenesis. To analyze transcriptional activity, a 1500 bp of the 5″UTR of cathelicidin gene CAMP was amplified with primers sense 5″-CACACAGCTAGCGGAACCCCTGGACAACGG-3″, (pGL 1500) and antisense 5″-GAGAGACTCGAGGTCTGCCTCCCTCTAGCC-3″ using human genomic DNA as a template. Primers were designed to introduce an NheI restriction site at the 5″end and an XhoI restriction site at the 3″end of the amplicon. The amplification product was cloned into the TOPO vector (Invitrogen) and transformed into E. coli TOP10 OneShot competent cells (Invitrogen). After DNA purification using the WizardPLus, SV Miniprep purification system (Promega, Madison, Wis.) the construct was digested with NheI and XhoI and then subcloned into the promoterless pGL3-basic firefly luciferase vector (Promega). The functional role of a previously described vitamin D responsive element (VDRE) for the transcription of CAMP was studied by site-directed mutagenesis of the VDRE. The VDRE at position -619 bp to -633 bp relative to the translation start site was deleted with sense 5″-AACTTCTGCTTCAGTGATTCTCAT-3″ and antisense 5″-ATGAGAATCACTGAAGCA GAAGTT-3″ primers. The resulting plasmid lacking the VDRE binding site cloned in the pGL3-basic vector was termed pGL-3 1500-VDRE. All resulting constructs were confirmed by sequencing. For transfection HaCat cells were seeded in 24 well plates (BD Biosciences) and used for transfection at 50 to 70% confluence. Cells were transfected with the indicated CAMP reporter plasmids and 0.1 μg of an internal-control Renilla luciferase expression plasmid (pRL-TK; Promega, Madison, Wis.) by using 1.5 μl of transfection reagent Fugene 6 (Roche) according to the manufacturer's instructions. Cells were stimulated with Malp-2 and 1,25D3 6 hours after transfection and incubated 24 h before harvesting with 50 μl of passive lysis buffer (Promega). Firefly luciferase activity from the CAMP pGL3 reporter vectors and Renilla luciferase activity were measured by the Dual Luciferase Assay system (Promega) in a luminometer (Optocomp I, MGM Instruments, Hamden, Conn.). Promoter activity was reported as the ratio between firefly and Renilla luciferase activities in each sample.

Immunostaining. After fixation in 2% PFA and subsequent washings in PBS, sections from skin tissue or epidermal constructs were blocked in 3% BSA in PBS for 30 min at RT and stained with anti-cathelicidin LL-37 primary antibody or preimmune serum. After washings in PBS, slides from epidermal constructs were re-probed with a FITC labeled goat anti-chicken antibody. Nuclei were detected with DAPI. After subsequent washings with PBS slides, were mounted in ProLong Anti-Fade reagent (Molecular Probes, Eugene, Oreg.) and evaluated with an Olympus BX41 microscope (Olympus, Melville, N.Y.). Paraffin embedded formalin fixed tissue sections were rehydrated in a series of toluene, ethanol, and PBS. Endogenous peroxidase activity was quenched by 30 min incubation in 0.3% H2O2 in water, and sections microwaved for 4 min in antigen retrieval solution (0.01M citric acid, 0.05M

NaOH, pH6.0). Sections were blocked with 2% goat serum in PBS 3% BSA then incubated with primary antibody (rabbit anti LL-37, or anti-TLR2 (Abcam)) in PBS, 0.1% bovine serum albumin. Sections were washed in PBS and detected with biotinylated secondary antibodies (Vector Laboratories, Burlingame, Calif.) and diaminobenzidine substrate (Sigma) per manufacturers' instructions.

Western Blot. Primary keratinocytes were stimulated with 1,25D3 (100 nM) or the vehicle for 24 hours and subsequently lysed in ice-cold lysis buffer (1% Triton-X in PBS containing proteinase inhibitors). After centrifugation, equal amounts of protein were mixed with loading buffer (0.25M Tris HCL, 10% SDS, 10% glycerol, 5% β-mercaptoethanol) and loaded onto a 10% Tris-Tricine gel (GeneMate, Kaysville, Utah). After separation, proteins were blotted onto a PVDF membrane (Millipore, Billerica, Mass.) and blocked in 5% milk (BioRad) in TBS 0.1% Tween20 for 1 h at RT. After washings in TBS 0.1% Tween20, membranes were stained with a rabbit polyclonal anti-CD14 (Abcam, Cambridge, Mass.) or an anti TLR2 antibody (eBioscience), washed again in TBS 0.1% Tween20 and reprobed with a HRP-coupled goat anti-rabbit antibody (DakoCytomation, Glostrup, Denmark). Stained protein was visualized using the Western Lightning® system (Perkin Elmer, Boston, Mass.).

Injury triggers a local increase in 1,25D3 signaling in skin.

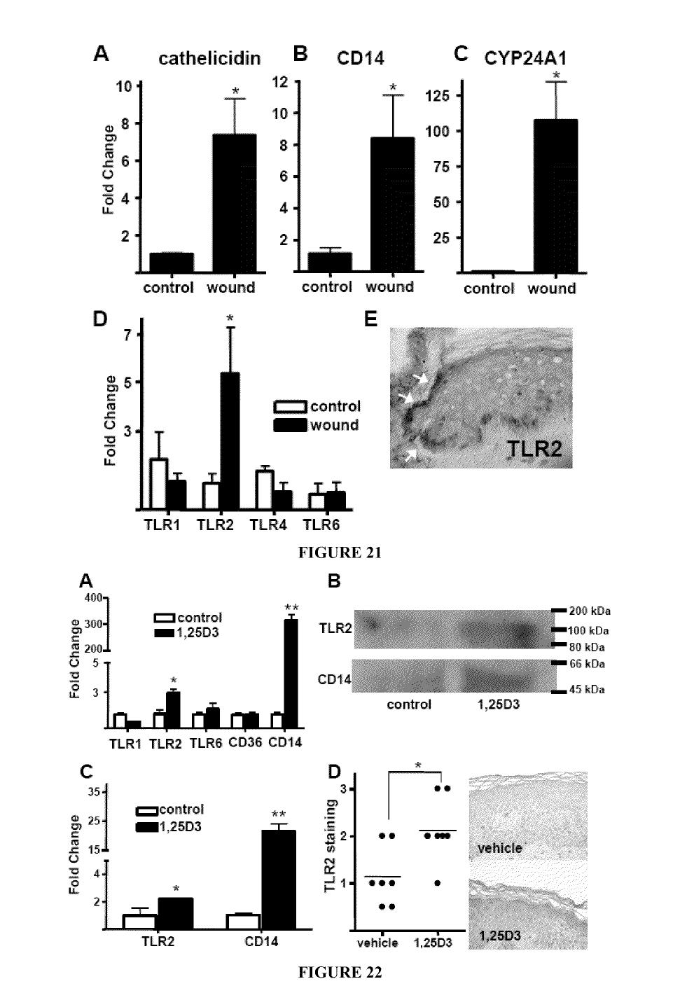

The function of elements of cutaneous innate immunity, such as the expression of Toll-like-receptors (TLRs) and the production of antimicrobial peptides (AMPs), are essential for control of infection. The expression of AMPs such as the cathelicidin gene CAMP, and the peptide LL-37 that it encodes, increases dramatically in epithelia after injury or infection. Finding that 1,25D3 can stimulate cathelicidin expression, and that cathelicidin increases following injury, prompted us to investigate if the expression of other elements of innate immunity, or 1,25D3 regulated genes, are induced after wounding. RNA extracted from skin wound biopsies obtained after sterile full thickness incision showed the expected increase in cathelicidin expression (FIG. 21 panel A). Coincident with this increase in cathelicidin the TLR cofactor CD14 and the 1,25D3 catabolic enzyme CYP24A1 were also induced (FIG. 21 panels B and C). Both of these genes were previously known to be inducible by 1,25D3, but not known to change during the response to injury. In addition, the expression of TLR2, not known to be influenced by 1,25D3, also increased as measured by transcript and protein abundance (FIG. 21 panels D and E). Expression of TLR1, TLR4 and TLR6 were not changed. 1,25D3 induces an increase in the expression of TLR2 and CD14. Finding that multiple diverse genes under the control of 1,25D3 are increased after injury suggested that the abundance or bioavailability of 1,25D3 may increase in the wound, and that these responses may be a previously unrecognised part of the human injury response.

Based on these observations it was hypothesized that TLR2 expression is controlled by 1,25D3. To test this, the response of cultured human keratinocytes to 1,25D3 was studied. Similar to the response seen following injury, CD14 and TLR2 mRNA and protein increased in response to 1,25D3 (FIG. 22 panels A and B). Other TLRs and the TLR coreceptor CD36 remained unchanged. TLR2 and CD14 transcript abundance was also induced by 1,25D3 in keratinocytes differentiated in epidermal constructs (FIG. 22 panel C). Skin of patients treated with topical 1,25D3 also showed increased expression of TLR2 (FIG. 22 panel D), and cathelicidin. This increase in TLR2 seen in patients following a single application of 1,25D3 did not induce clinical or histological evidence of inflammation. Therefore, these observations show that 1,25D3 can induce TLR2 in keratinocytes in vitro and in vivo, and that yet another element of the innate immune response is both induced by 1,25D3 and by injury. CYP27B1 is induced by injury and by soluble factors found in wounds.

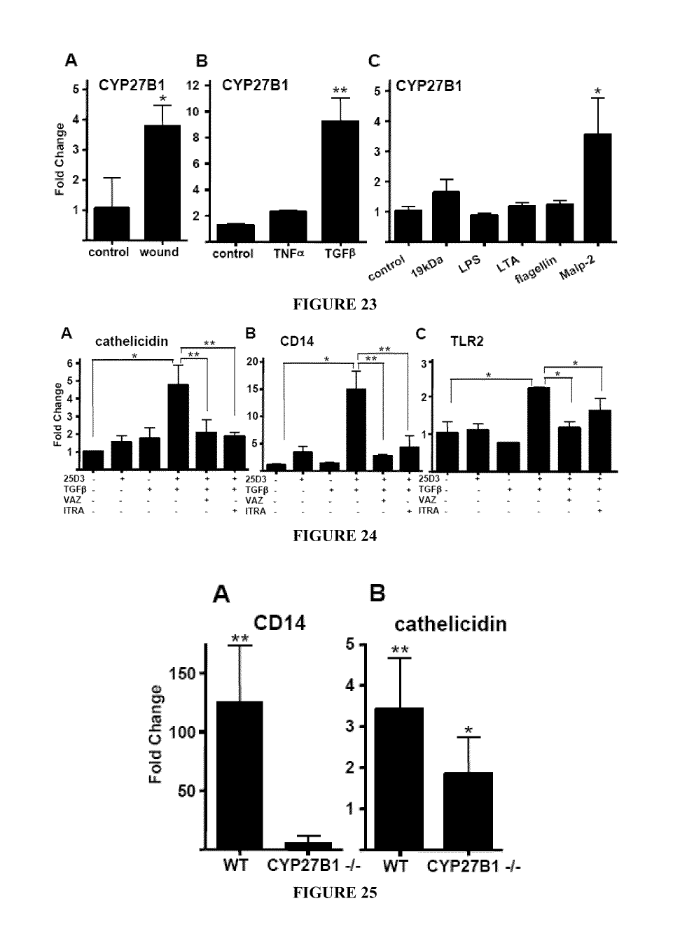

To test the hypothesis that the bioavailability of 1,25D3 is increased with wounding, examination of whether wounding or growth factors present in the wound influence CYP27B1, an enzyme capable of converting inactive 250H vitamin D3 (25D3) to active 1,25D3. In skin, the majority of vitamin D3 is present in an inactive pro-form, 7-dehydrocholesterol. Some soluble factors present in the wound such as IFNγ have been previously shown to influence 1,25D3 metabolism in monocytes and macrophages and reported to induce 1,25D3 production in non-differentiated keratinocytes. The regulation of this important enzyme following skin injury is unknown. In human skin, CYP27B1 expression was increased after wounding (FIG. 23 panel A). Cultured keratinocytes exposed to TGF also showed an increase in CYP27B1 expression while TNFα had no effect (FIG. 23 panel B). Also, similar to observations made in monocytes, CYP27B1 expression in keratinocytes could be induced by activation of TLR2 with the addition of Malp-2 (FIG. 23 panel C). These data show that injury leads to an increase in the expression of an enzyme capable of inducing a local increase in 1,25D3. This induction may occur secondary to the activity of factors present in the wound, such as IFNγ or TGFβ, or by microbial stimulation of TLR signaling. TGF induces cathelicidin, CD14 and TLR2 by CYP27B1 dependent activation of 1,25D3.

To directly test if the stimulation of CYP27B1 is responsible for the induction of the innate immune molecules now known to increase in wounds, keratinocytes were next exposed to TGF in the presence of 25D3. Prior studies with TGFβ, as well as several other molecules associated with wound repair, have not shown that these factors can regulate cathelicidin expression. However, it was hypothesized that by including 25D3 in culture similar to the accumulation of this precursor in normal skin, TGF mediated induction of CYP27B1 could then lead to activation of 1,25D3 and the subsequent induction of cathelicidin antimicrobial peptide and TLR expression. Neither 25D3 nor TGF alone affected expression, however their simultaneous addition induced cathelicidin, CD14 and TLR2 (FIG. 24 panels A-C). Specific inhibition of CYP27B1 by itraconazole, or addition of a VDR antagonist, blocked this effect (FIG. 24 panels A-C). These data show that the action of TGF and 25D3 was due to the enzymatic generation of 1,25D3.

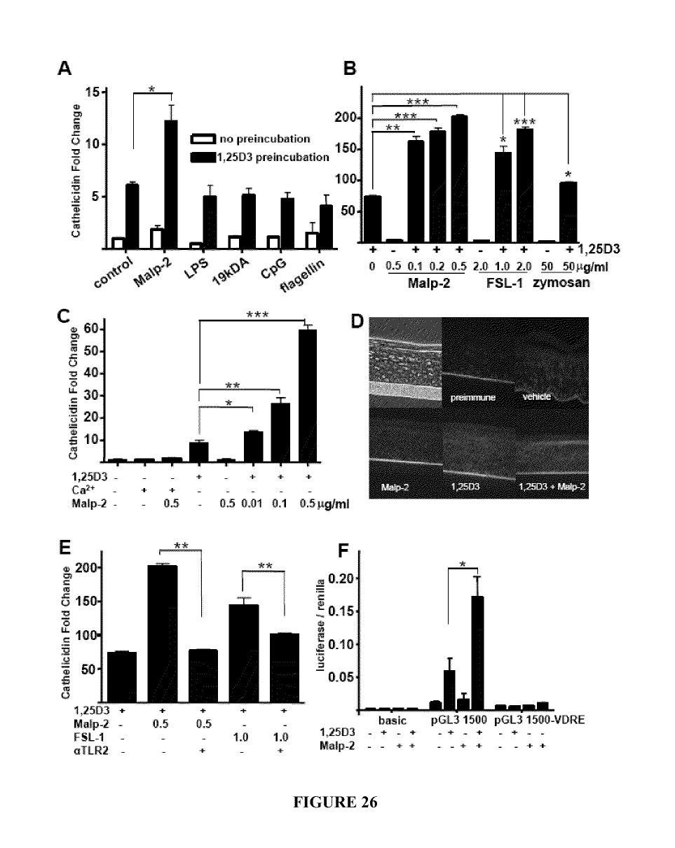

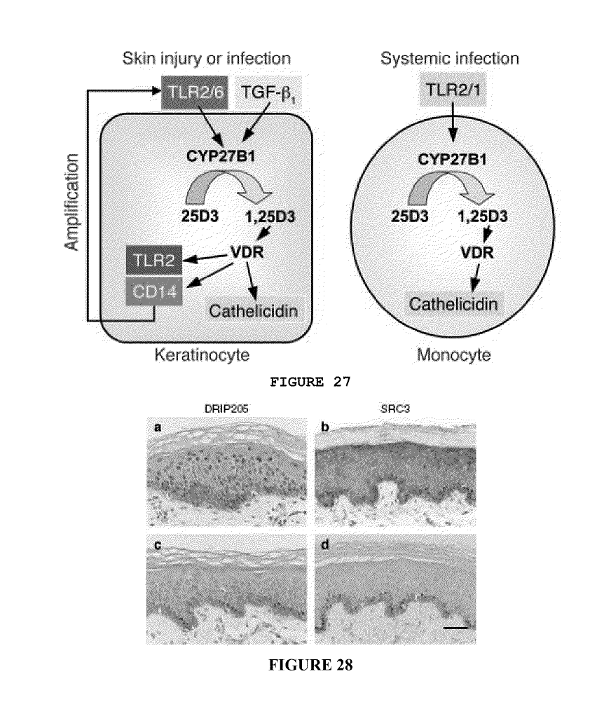

Induction of vitamin D3 regulated immune functions in skin wounds is mediated by CYP27B1 activation in vivo To further investigate the significance of increased CYP27B1 activation in skin injury wounds from mice lacking CYP27B1 enzyme were evaluated for induction of an innate immune response. In skin wounds, murine CD14 which is regulated by 1,25D3 was induced in wildype but not in animals lacking CYP27B1 (FIG. 25 panels A). In contrast, murine cathelicidin CRAMP, which is not regulated by 1,25D3, was increased in wounds in both animal strains (FIG. 25 panel B). These data underscore the importance of CYP27B1 activated 1,25D3 production and subsequent vitamin D3 triggered immune response in skin injury in vivo. 1,25D3 enhances TLR2/6 function by keratinocytes.

To explore the functional significance of TLR2 and CD14 induction by increased 1,25D3, keratinocytes were exposed to several TLR ligands and the antimicrobial response measured by evaluating the expression of cathelicidin. In contrast to cells grown under basal conditions, keratinocytes pretreated with low doses of 1,25D3 (0.1 nM; 24 hours) responded to Malp-2 but not to other TLR agonists such as LPS, CpG, 19 kDa lipopeptide and flagellin (FIG. 26 panel A). Other ligands of the TLR2,6 heterodimer in addition to Malp-2, such as zymosan and FSL-1, were also capable of inducing cathelicidin after keratinocytes were activated in the presence of 1,25D3 (100 nM) (FIG. 26 panel B). At low concentrations of 1,25D3 (0.1 nM) the significance of the enhanced responsiveness became even more apparent, with a dose dependent increase of cathelicidin expression of up to 60 fold greater than that seen in cells without 1,25D3 (FIG. 26 panel C).

To examine if this increased responsiveness to TLR2,6 ligands reflects a direct action of 1,25D3 on TLR function, or an indirect effect related to the changes in keratinocyte differentiation induced by 1,25D3, keratinocytes were studied following differentiation induced by stratification at the air-liquid interface or by increasing the concentration of extracellular calcium. Cultured keratinocyte monolayers incubated with high calcium concentrations (1.7 mM) as an alternative signal to induce terminal differentiation did not respond to TLR2,6 activation by Malp-2 (FIG. 26 panel C). This treatment was effective in stimulating differentiation as seen by increased expression of involucrin and altered cell morphology. Similarly, differentiated keratinocytes in epidermal constructs did not respond to Malp-2 in the absence of 1,25D3 but did after its addition (FIG. 26 panel D). The response to Malp-2 was due to TLR2 as a neutralizing antibody against TLR2 blocked the 1,25D3 dependent response to both Malp-2 and FSL-1 (FIG. 26 panel E). Also, supporting a direct effect of 1,25D3 on TLR2 function were observations of the transcriptional response of the cathelicidin promoter to Malp-2. Keratinocytes were transfected with CAMP promoter constructs including one with a site-directed disruption of the CAMP VDRE. In the presence of 1,25D3, promoter activity was responsive to Malp-2 in constructs that included the VDRE (FIG. 26 panel F). In the absence of 1,25D3 or the VDRE, activation of TLR2 had no significant effect on cathelicidin promoter activity. Thus, 1,25D3 both induces cathelicidin and enables TLR2 responsiveness to further increase expression of this antimicrobial peptide.

Wounding creates a breach in the otherwise intact physical and chemical cutaneous barrier against the outer environment. Shortly after a wound is created, epithelial cells become activated and produce multiple factors to protect against microbial invasion and trigger recruitment of leukocytes to further defend the wound. These epithelial derived molecules include AMPs, enzymes, cytokines and growth factors that initiate a complex and incompletely understood process of repair. Only after several hours to days do keratinocytes from the edge of the wound begin to migrate and reepithelialize the injury, thus reestablishing an effective physical barrier. Prior to final formation of this barrier it is essential for the unique wound micromilieu to protect the host from infection. Cathelicidin AMPs have been shown to be essential to this process, but the elements responsible for control of expression during injury were not known.

Multiple genes under the influence of vitamin D3, and TLR2 which was not previously known to be inducible by 1,25D3, were induced in injury. Results suggest that soluble factors in the wound such as TGF stimulate keratinocytes to increase the metabolic conversion of 250H vitamin D3 (25D3) to 1,25D3, thus driving the expression and function of cathelicidin and TLR2. The increase in TLR2 enables cells to respond to microbial stimulation and further enhance the AMP response if necessary, while also amplifying the generation of active vitamin D3. This elegant system of control of innate immunity by vitamin D3 was previously unknown, and adds a new element to the understanding of innate immune defence during wound repair.

Keratinocytes posses the complete enzymatic machinery to produce active 1,25D3 from stored inactive proforms. The final step of endogenous activation of vitamin D3 is hydroxylation of 25D3 by CYP27B1 (1α-hydroxylase). Cytokines such as IFNγ and TNFα have been identified over a decade ago to induce 1,25D3 production in non-differentiated keratinocytes. Evidence here suggests that the induction of TLR2, CD14 and cathelicidin was a direct consequence of this activity, which was induced by soluble factors such as TGFβ, resulting in increased local 1,25D3. TGF was observed to induce CYP27B1 and corresponded to the ability of inactive 25D3 to be converted. Lack of 25D3 as a precursor substrate, inhibition of CYP27B1 enzymatic activity, or blocking the vitamin D receptor, all inhibited the capacity of TGF to act in vitro. In vivo, mice lacking CYP27B1 enzyme fail to respond with an increase in 1,25D3 regulated CD14 upon skin injury. Further investigations to confirm that inhibition of CYP27B1 leads to an abnormal antimicrobial response in vivo would be helpful, but are hindered by the lack of currently available animal models to study this phenomenon since cathelicidin expression in mice is not under control of 1,25D3.

This distinct difference between humans and mice is currently unexplained, but may reflect an evolutionary adaptation to the nocturnal nature of mice compared with man. However, the data do show that topical administration to human volunteers of excess 1,25D3, in a manner that did not induce any evidence of an inflammatory response, did induce TLR2 and cathelicidin expression. Thus, the consequence of increasing CYP27B1 activity was confirmed to induce events that mimic the innate immune response to injury.

The observations suggest that activated vitamin D3 signaling during the wounding process initiates an antimicrobial response and increased sensitivity to microbial challenge through upregulation of TLR2 and the TLR cofactor CD14. This distinguishes keratinocytes from myeloid cells since in monocytes vitamin D3 downregulates TLR expression and triggers hyporesponsiveness to pathogen-associated molecular patterns. TLRs are important pattern recognition molecules and each recognizes distinct microbial components. For example, TLR2 senses peptidoglycans and lipoteichoic acid. A heterodimer of TLR2 with TLR1 or TLR6 is also able to sense triacetylated and diacetylated lipopeptides, respectively. Signaling through the TLR2,6 heterodimer requires the TLR coreceptors CD14 and CD36. Although 1,25D3 has been reported to induce CD14 in monocytes the invention also show that it can also induce CD14 in non-myeloid lineage cells such as keratinocytes. Supporting these data are prior observations that CD14 is also induced in skin during thermal injury. Thus, the findings show that following generation of 1,25D3 by activation of CYP27B1, CD14 and TLR2 are induced and recognition of microbial components such as Malp-2 or zymosan is enabled.

This study supports a novel role for vitamin D3 signaling in microbial recognition and the antimicrobial response during wound repair. As a consequence of these observations, future studies of TLR and AMP regulation have to consider the constitutively present elements of the tissue micromilieu such as precursor forms of vitamin D3. Without inclusion of 25D3 in these experiments, a molecule that is abundantly present in normal skin but typically eliminated from culture systems, the function of 1,25D3 would not have been detected. Based on these observations, it will also be important to investigate if a disturbed vitamin D3 metabolism leading to abnormal innate immune responses contributes to the development of diseases such as abnormal wound repair, psoriasis and atopic dermatitis that have altered expression of AMPs.

Hormonally active vitamin D3-1,25 dihydroxy vitamin D3 (1,25D3)—acts as a signalling molecule in cutaneous immunity by increasing pattern recognition through Toll-like receptor-2 (TLR2), and increasing the expression and function of antimicrobial peptides. Provided herein is data indicating that the actions of 1,25D3 on keratinocyte innate immune responses are influenced by histone acetylation and require the steroid receptor coactivator 3 (SRC3) that mediates inherent histone acetyltransferase (HAT) activity. SRC3 was detected in the suprabasal and granular layer of the skin, similar to cathelicidin expression. HAT activity was important to keratinocyte cathelicidin expression as the combination of histone deacteylase inhibitors (HDACi) (butyrate or trichostatin A) and 1,25D3 increased cathelicidin and CD14 expression and enhanced the antimicrobial function of keratinocytes against Staphylococcus aureus. This treatment, or activation of TLR2, also directly increased acetylation of histone 4. siRNA silencing of the vitamin D receptor or SRC3 blocked induction of cathelicidin and CD14 by 1,25D3. HDACi could not reverse this effect or influence cathelicidin in the absence of 1,25D3, suggesting that both are necessary for function. These studies demonstrate that the epigenetic control of gene transcription by histone acetylation is important for 1,25D3 regulated antimicrobial and TLR function of keratinocytes, essential elements of the innate immune response of the skin.

As noted throughout the present application, skin injuries result in the rapid induction of genes important for microbial recognition and antimicrobial defense by activated vitamin D3. In particular, 1,25D3 induces CD14 and TLR2 in keratinocytes enabling increased response to TLR activation. Simultaneously, vitamin D3 induces antimicrobial cathelicidin peptide expression strengthening the chemical antimicrobial shield produced by keratinocytes. The results of clinical studies indicate that topical treatment with 1,25D3 increases cathelicidin and TLR2 expression in healthy skin. These data indicate a role for vitamin D3 or its analogs as topical immune modulators.

The genomic effects of 1,25D3 are mediated by its nuclear hormone receptor, the vitamin D receptor (VDR). After binding of 1,25D3 the VDR binds to consensus sequences called vitamin D responsive elements (VDREs) in the promoter of target genes such as cathelicidin and CD14. The VDR subsequently recruits different coactivators to initiate transcriptional activity. It has been demonstrated that the VDR binds to coactivator complexes such as DRIPs (VDR-interacting proteins also called mediator) or SRCs (steroid receptor coactivators) during keratinocyte differentiation. During this process the coactivators have different functions. In proliferating keratinocytes DRIP binding to the VDR dominates, whereas in differentiated keratinocytes SRC binding is preferred. Furthermore, different genes prefer one type of complex compared to the others. For example, deletion of DRIP205 results in marked inhibition of keratin 1 expression, whereas deletion of SRC2 has little effect. In contrast, loricrin expression is highly dependent on DRIP205, as well as SRC2 and 3. The coactivator complexes have been shown to cycle on and off nuclear hormone response elements such as VDREs in alternating patterns suggesting that both types of complexes are required for optimal gene regulation. DRIP205 binds directly to VDR and the DRIP complex contains components of the transcription initiation complex linking it directly to the RNA polymerase machinery. SRC family members such as SRC3 form very different complexes with VDR. In particular they recruit a number of histone acetyltransferases such as CBP/p300 which by increasing histone acetylation open up the chromatin, thus facilitating access of transcription factors to the transcription start site. Reversible acetylation of histone proteins is generally correlated with active transcription, whereas deacetylation is linked to transcriptional repression.

Histones H2A, H2B, H3 and H4 are the core subunits that comprise the nucleosome, the smallest unit of eukaryotic chromatin. The amino termini of these four histones are accessible to post-translational modifications directly influencing gene transcription. Both histone tails and globular domains are subject to posttranslational modifications, and these modifications have different functions. The modifications include methylation of arginine residues; methylation, acetylation, ubiquitination, ADP-ribosylation, and sumolation of lysines; and phosphorylation of serines and threonines. With the exception of methylation, histone modifications result in a change in the net charge of nucleosomes, which may act to relax inter- or intranucleosomal DNA-histone interactions. Increased histone acetylation at the promoter region has been linked to active transcription. Recently, Pokholok and coworkers demonstrated that increased acetylation of H3 and H4 correlates with increased transcription rate (Pokholok et al, Cell 122(4):517-27, 2005). The locations of these chromosomal modifications are tightly regulated by histone acetyltransferases (HATs) such as the SRC3/p300 complex and histone deacetylases (HDACs) and are crucial for their effect on transcription.

The short-chain fatty acid butyrate and trichostain A (TSA) increase acetylation of histone H4 by inhibition of HDAC activity. This in turn results in induction or repression of genes in various cells including keratinocytes. Changes in histone acetylation may “shape” the response to additional stimuli such as growth factors.

The effects of vitamin D3 on keratinocyte innate immune function indicate that VDR coactivators such as SRC3 and DRIP205, which participate in distinct ways in mediating vitamin D dependent keratinocyte differentiation, may also be involved in regulation of cutaneous innate immunity.

The localization of DRIP205 and SRC3 in human skin was determined using immunohistochemistry. DRIP205 was present throughout the epidermis (FIG. 28 panel A). The expression of SRC3 was distinct from DRIP205. SRC3 was strongly expressed in the differentiated keratinocytes in the superficial epidermis with expression increasing gradually from spinous to granular layer (FIG. 28 panel B). Also, while DRIP205 staining was mainly nuclear, SRC3 was detected in both cytoplasm and the nucleus in keratinocytes. This expression of the VDR coactivator SRC3 is consistent with observations showing that cathelicidin antimicrobial peptide (hCAP18/LL-37) is induced by the activity of 1,25D3. Thus, SRC3 and cathelicidin show a similar expression pattern that is distinct from another VDR coactivator, DRIP205.

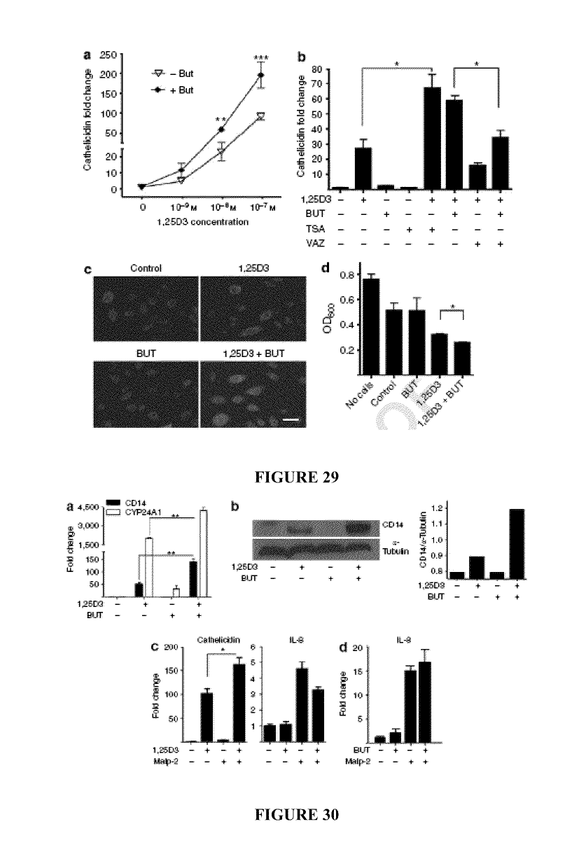

SRC3 complexes possess histone acetyltransferase activity unlike DRIP complexes. Normal keratinocytes were treated with butyrate to increase histone acetylation by inhibition of histone deacetylases (HDACs) in order to determine if histone acetylation influences antimicrobial peptide expression in skin. Cells were exposed to increasing 1,25(OH)₂ vitamin D3 (1,25D3) levels at a constant butyrate concentration (2 mM) and the expression of cathelicidin (hCAP18/LL-37) mRNA measured by quantitative real-time PCR (FIG. 29 panel A). The addition of butyrate alone did not change cathelicidin transcript abundance in keratinocytes. However, HDAC inhibition significantly amplified cathelicidin expression in keratinocytes in the presence of 1,25D3. In the presence of 10⁻⁷ M 1,25D3, cathelicidin increased over 200-fold in the presence of butyrate but only about 75-fold in its absence. 24 hours after treatment with butyrate alone or in combination with 1,25D3 no change was seen in cell morphology, cell death or apoptosis as measured by microscopic inspection, LDH release and Annexin V /propidium iodide staining, respectively. Trichostatin A—another HDAC inhibitor structurally unrelated to butyrate—showed similar effects and also significantly increased 1,25D3 induced cathelicidin (FIG. 29 panel B). Treatment with the VDR antagonist VAZ decreased the capacity of the HDAC inhibitor to increase cathelicidin (FIG. 29 panel C), indicating that increasing histone acetylation augments VDR transcriptional activity but that cathelicidin expression was still dependent on the action of the VDR. In addition to the increase in mRNA, an increase in cathelicidin peptide expression was seen by immunostaining of keratinocytes treated with butyrate and 1,25D3 (FIG. 29 panel C). Furthermore, HDAC inhibition increased the inherent antimicrobial activity of 1,25D3 stimulated keratinocytes. Protein extracts from cells treated with 1,25D3 and butyrate displayed maximal ability to suppress the growth of Staphylococcus aureus AmprF (FIG. 29 panel D). HDACi and 1,25D3 did not show antimicrobial activity themselves. These findings are consistent with an increase in proteolytic activation of cathelicidin in addition to the observed increase in mRNA and protein expression.

To investigate if HDAC inhibition specifically increased cathelicidin, other genes in keratinocytes that are regulated by vitamin D3 were investigated. Similar to cathelicidin, CD14 and CYP24A1 transcript abundance was strongly upregulated by 1,25D3 stimulation and further enhanced by inhibition of HDAC with butyrate (FIG. 3 panel a). An increase in CD14 protein expression was also seen in similarly treated keratinocytes (FIG. 30 panel B).

The expression of the CXCL chemokine IL-8 was measured to determine if an increase in histone acetylation would also induce other immune response genes in keratinocytes. The activation of TLR2/6 by Malp-2 in the presence of 1,25D3 is known to increase cathelicidin, but Malp-2 alone has no effect (FIG. 30 panel C). In contrast, keratinocyte IL-8 expression increased when stimulated with only Malp-2 and was not enhanced by 1,25D3 or butyrate (FIG. 30 panel C and panel D). These data indicate that increased histone acetylation selectively enhances expression of the 1,25D3 dependent genes cathelicidin and CD14, but does not increase IL-8 in keratinocytes.

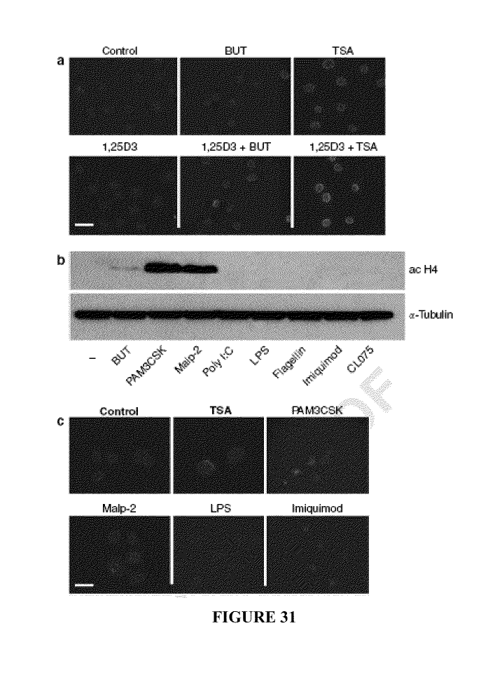

The abundance of acetylated histone was directly measured to confirm the inhibitory effects of butyrate and TSA on HDACs. Immunofluorescence staining demonstrated that normal keratinocytes stimulated with HDACi increased histone H4 acetylation localized to the nuclei (FIG. 31 panel A). Histone H4 acetylation was further increased when keratinocytes were stimulated with HDACi in the presence of 1,25D3 which was consistent with the observed increase in transcriptional activity. To determine if changes in histone acetylation occur upon stimulation of keratinocytes with TLR ligands NHEK were stimulated with different TLR ligands and histone acetylation was assessed. Butyrate treatment increased acetylation of histone H4 (FIG. 31 panel b, lane 2). Exposure to Malp-2 or PAM3CSK to activate TLR2/6 or TLR2/1 respectively also increased histone acetylation (FIG. 31 panel B, lane 3 and 4). Ligands of other TLRs including; Poly(I:C)-TLR3, LPS-TLR4, Flagellin-TLR5, Imiquimod-TLR7 and CL075-TLR7/8, did not change histone acetylation in primary keratinocytes.

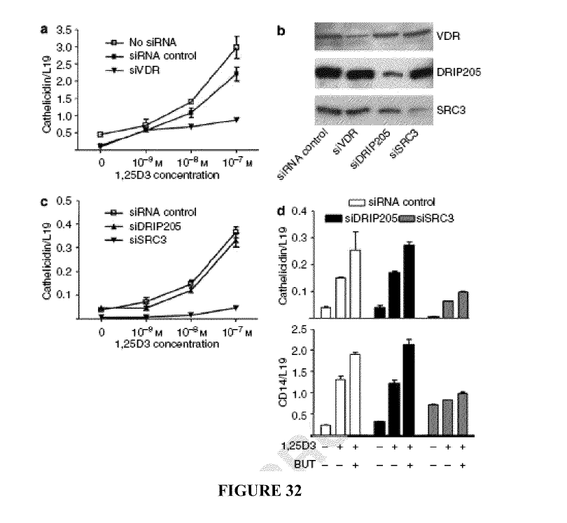

To further examine how cathelicidin expression is influenced by histone acetylation, the role of the VDR and its two distinct coactivators, DRIP205 and SRC3, cells were examined using siRNA silencing technology. Among multiple DRIP subunits, DRIP205 was targeted because it directly binds to VDR through its NR boxes. From the two SRC family members, SRC3 was selected because it had previously been shown to increase VDR transactivation. The results demonstrate that silencing of the VDR by siRNA blocked induction of cathelicidin by 1,25D3 (FIG. 32 panel A) and CD14. This effect corresponded with a 70% decrease in VDR protein expression as displayed in FIG. 32 panel B. Blocking of VDR coactivator DRIP205 had no effect on 1,25D3 induced cathelicidin (FIG. 32 panel C) despite inhibition of DRIP205 expression confirmed by immunoblotting (FIG. 32 panel B). In contrast, inhibition of SRC3 expression blocked vitamin D3 induced cathelicidin expression (FIG. 31 panel C) and CD14 (FIG. 32 panel D). Increasing histone acetylation by butyrate did not restore cathelicidin and CD14 induction by 1,25D3 when SRC3 was silenced (FIG. 32 panel D). Again, blocking DRIP205 had no effect on cathelicidin and CD14 induction by 1,25D3 in the presence or absence of HDAC inhibitors (FIG. 32 panel D). These results indicate that histone acetyltransferase activity is mediated by the SRC3 complex.

In response to TLR activation or injury, cutaneous vitamin D3 metabolism is altered and hormonally active 1,25D3 generated to serve as a signaling molecule in a sequence of rapid defense responses in keratinocytes and monocytes. The present data indicate that the presence of 1,25D3 increases CD14 and TLR2 expression in vitro and in vivo and enables pattern recognition leading to amplification of the antimicrobial response. Activated 1,25D3 generated in the skin or applied externally initiates an antimicrobial response and increases sensitivity to microbial challenge in skin. The data provided herein further indicate that increased histone acetylation enhanced 1,25D3 induced innate defense genes and increased antimicrobial activity in keratinocytes. This effect was mediated through the VDR and dependent on SRC3. Blocking or silencing of the VDR or SRC3 inhibited 1,25D3 induced cathelicidin and CD14 gene expression. HDAC inhibition by butyrate could not reverse this effect. These data indicate that chromatin opening through histone acetyltransferase activity mediated by the SRC3 complex is necessary before the effects of VDR mediated transcription can be enhanced by histone deacetylase inhibition. These data provide the first example of gene induction by 1,25D3 independent of DRIP205, suggesting that unraveling of the chromatin via histone acetylation plays an important role than activation of the RNA polymerase via this mediator.

TLR2 ligands such as Malp-2 or PAM3CSK were shown here to greatly increase the acetylation of histone 4 in keratinocytes. When cells were stimulated with both TLR2/6 ligands and 1,25D3 they show the greatest increase in cathelicidin (Schauber, Dorschner et al. 2007). This response is similar to the increased cathelicidin seen after HDAC and 1,25D3 treatment. Combined with observations of the inhibition of expression by suppression of SRC3, these data strongly suggest histone acetylation is a modifier of 1,25D3 dependent effects. However, increased histone acetylation alone did not greatly induce cathelicidin or influence expression of other immune response genes such as IL-8. Since TLR2 activation by Malp-2 or PAM3CSK can induce IL-8, this suggests histone acetylation cannot be the sole mechanism for the induction of either antimicrobial peptide expression or IL-8 production. These findings do however suggest that histone acetylation in keratinocytes can explain the amplification of an antimicrobial response to TLR2 ligands in the presence of 1,25D3 (Schauber, Dorschner et al. 2007). Other cell types have distinct responses. TLR mediated gene expression in macrophages and dendritic cells can be modified by HDAC inhibition (Brogdon, Xu et al. 2007) and colonic epithelial cells can be induced to increase cathelicidin by HDAC alone (Schauber, Dorschner et al. 2006). These observations show that the nature of a response to histone acetylation will be cell-type and gene specific.

The data reveal a novel mechanism in the regulation of innate immune function in skin. These data further provide a basis for treating disorders associated with the skin. Various HDAC inhibitors have been evaluated preclinically as therapeutic agents with potential roles in cancer treatment or as anti-inflammatory drugs. However, the present studies are the first to identify a therapeutic application for HDAC inhibitors in strengthening and fine tuning innate immune function in skin.

For example, HDACi may be used in combination with 1,25D3 or its analogs to prevent or treat cutaneous infections. Alternatively, HDACi treatment may be combined with widely used UVB irradiation which stimulates endogenous production of 1,25D3 to increase the innate immune defense barrier. Recently, the 1,25D3 analog Maxacalcitol (1,250H₂-22-oxacalcitriol) was used to treat viral infections of the skin. Currently, vitamin D3 analogs are also successfully used in the treatment of non-infectious inflammatory skin diseases such as psoriasis. The results provided herein indicate that in combination therapy with HDACi, 1,25D3 dosages may be lowered thereby minimizing the risk of 1,25D3 mediated side. Alternatively or additionally, the beneficial activity of existing topical 1,25D3 treatments may be enhanced by such a combination.

A number of embodiments of the invention have been described. Nevertheless, it will be understood that various modifications may be made without departing from the spirit and scope of the invention. Accordingly, other embodiments are within the scope of the following claims.

What is claimed is:

1. A method for modulating an innate immune response in a target tissue, the method comprising contacting the target tissue with 1,25(OH)₂ vitamin D3, wherein said contacting induces the expression of at least one nucleic acid sequence encoding a polypeptide that modulates the innate immune response.

2. The method of claim 1, wherein the target tissue is contacted with 1,25(OH)₂ vitamin D3 by inducing the production of 1,25(OH)₂ vitamin D3 from a precursor molecule at the target tissue.

3. The method of claim 2, wherein the precursor molecule comprises vitamin D3.

4. The method of claim 2, wherein the production of 1,25(OH)₂ vitamin D3 is induced by 1-alpha hydroxylase (CYP27B1) and/or 25 hydroxylase (CYP27A1).

5. The method of claim 1, wherein the nucleic acid sequence is operably associated with a regulatory sequence comprising at least one vitamin D responsive element (VDRE).

6. The method of claim 5, wherein the nucleic acid sequence encodes a microbial pattern recognition receptor (PRR).

7. The method of claim 6, wherein the microbial pattern recognition receptor (PRR) comprises CD14 or toll-like receptor 2 (TLR2).

8. The method of claim 1, wherein the nucleic acid sequence encodes a polypeptide comprising a cathelicidin functional peptide that is 16-36 amino acids in length and contains the sequence NH₂—X₁X₂X₃X₄X₅X₆IKX₇FX₈X₉X₁₀LX₁₁P—COOH (SEQ ID NO:1), wherein X₁, X₂, and X₆ are individually K or R; wherein X₃ is I or K; wherein X₄ is V or G; wherein X₅ is Q or R; wherein X₈, X₉, X₁₀, and X₁₁ are each individually any amino acid; wherein X₈ is L or F and wherein the polypeptide comprises antimicrobial and/or antiviral activity.

9. The method of claim 1, wherein the target tissue comprises keratinocytes.

10. The method of claim 1, wherein the target tissue comprises a skin disorder selected from a wound, a bacterial infection, a viral infection, a diabetic ulcer, a chronic skin ulcer, acne or acne rosacea.

11. The method of claim 1, wherein the modulating comprises stimulation of the innate immune response in a target tissue.

12. The method of claim 1, wherein the 1,25(OH)₂ vitamin D3 induces expression of a nucleic acid sequence by forming a complex with the vitamin D receptor, wherein the complex binds to a vitamin D responsive element (VDRE) associated with a regulatory element operably associated with the nucleic acid sequence.

13. The method of claim 12, wherein the complex further associates with VDR interacting proteins or steroid receptor co-activators (SRCs).

14. The method of claim 1, wherein the 1,25(OH)₂ vitamin D3 induces the expression of at least one nucleic acid sequence by modifying the histone acetylation state of a regulatory element operably associated with the nucleic acid sequence.

15. A method for modulating the innate immune response of a subject, the method comprising administering to the subject in need of such treatment a composition, or a pharmaceutically acceptable salt thereof, containing a therapeutically effective amount of a compound that comprises:
a) 1,25(OH)₂ vitamin D3;
b) a 1,25(OH)₂ vitamin D3 precursor; or
c) an agent that increases the production of 1,25(OH)₂ vitamin D3 at the site of the target tissue,
wherein expression of at least one nucleic acid sequence encoding a polypeptide that modulates the innate immune response is induced in the subject.

16. The method of claim 15, wherein the composition further comprises an antimicrobial agent.

17. The method of claim 16, wherein the antimicrobial agent is an antibiotic.

18. The method of claim 15, wherein modulating the innate immune response effectively treats a systemic infection or localized infection.

19. A method for promoting wound healing, the method comprising:
a) contacting the site of a wound with 1,25(OH)₂ vitamin D3; or
b) inducing the expression of 1,25(OH)₂ vitamin D3 at the site of the wound.

20. The method of claim 21, wherein the contacting promotes wound healing by modulating an innate immune response in the tissue associated with the wound site.

21. A method of increasing the expression of a microbial pattern recognition receptor (PRR) in a cell, the method comprising contacting the cell with a composition comprising:
a) vitamin D;
b) 1,25(OH)₂ vitamin D3; or
c) a compound that increases the production of 1-alpha hydroxylase (CYP27B1) in the cell.

22. The method of claim 21, wherein the microbial pattern recognition receptor (PRR) is CD14 or toll-like receptor 2 (TLR2).

23. A pharmaceutical composition, or pharmaceutically acceptable salt thereof, comprising 1,25(OH)₂ vitamin D3 or a precursor to vitamin D3.

24. The pharmaceutical composition of claim 23 further comprising an antimicrobial agent.

25. The pharmaceutical composition of claim 23 formulated for topical administration.


SEQUENCE LISTING (22 sequences)

SEQ ID NO: 1 - protein, 16 aa, Homo sapiens
  X (1)..(6): K or R
XXXXXXIKXFXXXLXX

SEQ ID NO: 2 - protein, 11 aa, Homo sapiens
KRIVQRIKDVF

SEQ ID NO: 3 - protein, 8 aa, Homo sapiens
RKSKEKIG

SEQ ID NO: 4 - protein, 8 aa, Homo sapiens
KSKEKIGK

SEQ ID NO: 5 - DNA, 739 bp, Homo sapiens
taaagcaaaccccagcccacaccctggcaggcagccagggatgggtggatcaggaaggctcctggttgggcttttgcatcaggctcaggctgggcataaaggaggctcctgtgggctagagggaggcagacatggggaccatgaagacccaaagggatggccactccctggggcggtggtcactggtgctcctgctgctgggcctggtgatgcctctggccatcattgcccaggtcctcagctacaaggaagctgtgcttcgtgctatagatggcatcaaccagcggtcctcggatgctaacctctaccgcctcctggacctggaccccaggcccacgatggatggggacccagacacgccaaagcctgtgagcttcacagtgaaggagacagtgtgccccaggacgacacagcagtcaccagaggattgtgacttcaagaaggacgggctggtgaagcggtgtatggggacagtgaccctcaaccaggccaggggctcctttgacatcagttgtgataaggataacaagagatttgccctgctgggtgatttcttccggaaatctaaagagaagattggcaaagagtttaaaagaattgtccagagaatcaaggattttttgcggaatcttgtacccaggacagagtcctagtgtgtgccctaccctggctcaggcttctgggctctgagaaataaactatgagagcaatttcaaaaaaaaaaaaaaaaaaaaaaaaa

SEQ ID NO: 6 - protein, 170 aa, Homo sapiens
MKTQRNGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIDDFLRNLVPRTES

SEQ ID NO: 7 - DNA, 519 bp, Homo sapiens
atgcagttccagagggacgtcccctccctgtggctgtggcggtcactatcactgctgctgctactgggcctggggttctcccagacccccagctacagggatgctgtgctccgagctgtggatgacttcaaccagcagtccctagacaccaatctctaccgtctcctggacctggatcctgagccccaaggggacgaggatccagatactcccaagtctgtgaggttccgagtgaaggagactgtatgtggcaaggcagagcggcagctacctgagcaatgtgccttcaaggaacagggggtggtgaagcagtgtatgggggcagtcaccctgaacccggccgctgattcttttgacatcagctgtaacgagcctggtgcacagccctttcggttcaagaaaatttcccggctggctggacttctccgcaaaggtggggagaagattggtgaaaagcttaagaaaattggccagaaaattaagaatttttttcagaaacttgtccctcagccagagtag

SEQ ID NO: 8 - protein, 173 aa, Mus musculus
MQFQRDVPSLWLWRSLSLLLLLGLGFSQTPSYRDAVLRAVDDFNQQSLDTNLYRLLDLDPEPQGDEDPDTPKSVRFRVKETVCGKAERQLPEQCAFKEQGVVKQCMGAVTLNPAADSFDISCNEPGAQPFRFKKISRLAGLLRKGGEKIGEKLKKIGQKIKNFFQKLVPQPEQ

SEQ ID NO: 9 - protein, 172 aa, Canis lupus
METQKDSPSLGRWSLLLLLLGLVITPAASRALSYREAVLRAVNGFNQRSSEENLYRLLQLNSQPKGDEDPNIPKPVSFTVKETVCPKTTQQPLEQCGFKDNGLVKQCEGTVILDEDTGYFDLNCDSILQVKKIDRLKELITTGAQKIGKKIRRIGQRIKDFLKNLQPREEKS

SEQ ID NO: 10 - protein, 172 aa, Sus scrofa
METQRASLCLGRWSLWLLLLALVVPSASAQALSYREAVLRAVDRLNEQSSEANLYRLLELDQPPKADEDPGTPKPVSFTVKETVCPRPTRQPPELCDFKENGRVKQCVGTVTLNPSIHSLDISCNEIQSVRRRPRPPYLPRPRPPPFFPPRLPPRIPPGFPPRFPPRFPGKR

SEQ ID NO: 11 - protein, 176 aa, Capra aegagrus
METQGASLSLGRWSLWLLLLGLVVPLASAQALSYREAVLRAVGQLNERSSEANLYRLLELDPAPNDEVDPGTRKPVSFTVKETVCPRTTQQPPEECDFKENGLVKQCVGTVTLDPSNDQFDINCNELQSVRFRPPIRRPPIRPPFNPPFRPPVRPPFRPPFRPPFRPPIGPFPGRR

SEQ ID NO: 12 - protein, 11 aa, Artificial Sequence
  linker moity
SSLLEKGLDGA

SEQ ID NO: 13 - protein, 5 aa, Artificial Sequence
  Dermicidin Peptide fragment from Homo sapiens
SSLLE

SEQ ID NO: 14 - protein, 37 aa, Artificial Sequence
  Peptide Fragment from LL-37
LLGDFFRKSKEKIGKEFKRIVQRIKDFLRNLVPRTES

SEQ ID NO: 15 - protein, 31 aa, Artificial Sequence
  Peptide fragment from LL-37
RKSKEKIGKEFKRIVQRIKDFLRNLVPRTES

SEQ ID NO: 16 - protein, 30 aa, Artificial Sequence
  Peptide fragment from LL-37
KSKEKIGKEFKRIVQRIKDFLRNLVPRTES

SEQ ID NO: 17 - protein, 20 aa, Artificial Sequence
  Peptide fragment from LL-37
KRIVQRIKDFLRNLVPRTES

SEQ ID NO: 18 - protein, 47 aa, Homo sapiens
SSLLEKGLDGAKKAVGGLGKLGKDAVEDLESVGKGAVHDVKDVLDSV

SEQ ID NO: 19 - DNA, 30 bp, Artificial Sequence
  Oligonucleotide Primer
cacacagctagcggaacccctggacaacgg

SEQ ID NO: 20 - DNA, 30 bp, Artificial Sequence
  Oligonucleotide Primer
gagagactcgaggtctgcctccctctagcc

SEQ ID NO: 21 - DNA, 24 bp, Artificial Sequence
  Oligonucleotide Primer
aacttctgcttcagtgattctcat

SEQ ID NO: 22 - DNA, 24 bp, Artificial Sequence
  Oligonucleotide Primer
atgagaatcactgaagcagaagtt